(19) United States
(12) Patent Application Publication
(10) Pub. No.: US 2024/0269296 A1
(43) Pub. Date: Aug. 15, 2024

(54) ANTIOXIDANT NUCLEOLIPID PRODRUG, PHARMACEUTICAL COMPOSITION FOR ADMINISTRATION THEREOF, AND THERAPEUTIC USES THEREOF

(71) Applicants: UNIVERSITE DE BORDEAUX, BORDEAUX (FR); CENTRE NATIONAL DE LA RECHERCHE SCIENTIFIQUE, PARIS (FR); INSERM (INSTITUT NATIONAL DE LA SANTE ET DE LA RECHERCHE MEDICALE), PARIS (FR); CENTRE HOSPITALIER UNIVERSITAIRE DE BORDEAUX, Talence (FR)

(72) Inventors: Valérie Desvergnes, Villenave d'Ornon (FR); Sylvie Crauste-Manciet, Bordeaux (FR); Arthur Klufts-Edel, Horbourg- Wihr (FR)

(21) Appl. No.: 18/560,511

(22) PCT Filed: May 13, 2022
(86) PCT No.: PCT/EP2022/063064

§ 371 Date: Nov. 13, 2023

(30) Foreign Application Priority Data
May 14, 2021 (FR) 2105088

(51) Int. Cl. A61K 47/54; A61P 17/00

(52) U.S. Cl. CPC A61K 47/549; A61K 47/543; A61P 17/00

(57) ABSTRACT

A prodrug formed by a compound including a pharmaceutically active antioxidant molecule which is covalently coupled, via a bond comprising at least one carboxylic ester group, to a nucleoside devoid of free hydroxyl and primary amine functions, comprising a ribose or deoxyribose unit covalently bonded to a nucleic base, this nucleoside bearing at least one lipidising group. A pharmaceutical composition which is particularly useful for therapeutic uses contains this prodrug in a nanoemulsion, in which the prodrug is contained in the lipophilic phase.

The present invention relates to the field of therapeutic applications of pharmaceutically-active molecules with an antioxidant activity.

More particularly, the present invention relates to a prodrug based on a pharmaceutically-active antioxidant molecule, as well as to a pharmaceutical composition containing such a prodrug. The invention also relates to therapeutic applications of this prodrug and of this pharmaceutical composition.

Antioxidant compounds are widely used in the medical field for the prevention and treatment of many pathologies, taking advantage of their ability to inhibit the oxidation of other molecules and in particular to protect them from the action of reactive oxygen species, in particular free radicals, and from oxidative stress, involved in the development of these pathologies.

In particular, the beneficial effect of antioxidant compounds has been demonstrated in the context of the repair of cutaneous burns or for the preventive or curative treatment of ophthalmic diseases, such as age-related macular degeneration (AMD) or cataract. The publication by Laskin et al., Annals of the New York Academy of Sciences, 2010, 92-100, describes the protective effect of the molecules targeting the oxidative stress pathways against the burns induced by the mustard gas.

It has also been described in the prior art, for example in the publication by Delanty and Dichter, Arch. Neurol. 2000, 57, 1265-1270, that antioxidant compounds allow preventing, delaying or improving numerous neurological disorders. The publication by Leeuw et al., Alzheimer's Dement. 2020, DOI: 10.1002/trc2.12021, the publication by Leeuw et al., in Journal of Alzheimer's disease, 2020, 619-627, the publication by Ye Feng et al., Oxidative Medecine and Cellular Longevity, 2012, 1-17, or else the publication by Moneim, Current Alzheimer Research, 2015, 12, 335-349, more specifically indicate that antioxidants, and in particular for some of them vitamin E, and in particular α-tocopherol, contribute to the prevention and treatment of Alzheimer's disease. The delivery of the pharmaceutically-active molecules in general, and of antioxidant molecules in particular, to their pharmacological targets is one of the major concerns of medicinal chemistry and galenic chemistry. Thus, numerous systems have been proposed by the prior art for conveying the active ingredients in the organism, and promoting passage thereof through the biological membranes, while protecting them against a possible degradation before having reached their target. In this respect, mention may be made, as examples, of nanocarriers, dendrimers, therapeutic capsules, etc.

The present invention aims to provide a system allowing carrying the pharmaceutically-active antioxidant molecules efficiently within the organism, and in particular promoting passage thereof through the biological membranes, preferably including through the blood-brain membrane, up to the inside of the neurons, regardless of whether the antioxidant molecules have a lipophilic or hydrophilic nature, and while ensuring that they remain stable until they reach their target.

To this end, the present Inventors have been interested in the principle of prodrugs, according to which a pharmaceutically-active molecule is conjugated to a compound acting as a vector for transport thereof in the organism, from which vector it is separated afterwards, by a biological process, to be released into the organism in its pharmaceutically-active form.

The document WO 2016/057825 describes prodrugs for intracellular delivery of a monophosphorylated nucleoside, wherein a nucleoside or a nucleoside analogue such as 5-azacytidine is conjugated to vitamin E via one phosphodiester or phosphoramidate bond.

The publication by Sinokrot Hanadi et al. Molecules, 2017, 1736 describes different strategies for improving the delivery of nucleoside-type antiviral agents. In quite another field, that of the delivery of antioxidant molecules, the present inventors have discovered that the use of a vector having a specific chemical structure, of the type derived from a nucleoside made lipidic by fixation of a lipid group, proves to be a particularly good absorption promoter so that it enables the passage of the antioxidant molecules of both lipophilic and hydrophilic nature through the biological membranes. Advantageously, this particular vector also allows stabilising the antioxidant molecules, by avoiding degradation thereof, in particular oxidation thereof, in the organism, while maintaining/conferring good bioavailability thereof, enabling in particular their oral administration.

Thus, according to a first aspect, the present invention relates to a prodrug consisting of:
a compound, so-called active compound, comprising:
a pharmaceutically-active antioxidant molecule covalently coupled, by a bond comprising at least one carboxylic ester group, to a nucleoside comprising a ribose or deoxyribose unit covalently bonded to a nucleic base,
said nucleoside bearing at least one group, referred to as a lipidising group, selected from among —R and —CO—R groups, wherein R represents a hydrocarbon radical, preferably a C1-C55, preferably C1-C25 and more preferably C10-C25, hydrocarbon radical, linear, branched and/or cyclic, saturated and/or unsaturated, aromatic or not, optionally substituted, optionally interrupted by one or more heteroatom(s), such as oxygen, nitrogen and sulphur atoms, and/or by one or more group(s) comprising at least one heteroatom or at least one group comprising at least one heteroatom, R however comprising no hydroxyl, primary amine, secondary amine, phosphate or thiol group, said hydrocarbon radical possibly including one cycle or several cycles, optionally one or more heterocycle(s) comprising one or more heteroatom(s) such as oxygen, nitrogen or sulphur atoms, said cycles being optionally condensed,
the lipidising group being optionally linked by covalent bonding simultaneously to two distinct atoms of the nucleoside,
or one of the pharmaceutically-acceptable salts of this active compound. Furthermore, in the active compound according to the invention:
each of the hydroxyl groups of said ribose or deoxyribose unit not involved in a bond to the antioxidant molecule or to a lipidising group is substituted by a protective group of a hydroxyl function fixed to the oxygen atom of said hydroxyl group and selected from among linear, branched and/or cyclic, saturated and/or unsaturated, hydrocarbon radicals, aromatic or non-aromatic, optionally substituted, optionally interrupted by one or more heteroatom(s), such as oxygen, nitrogen and sulphur atoms, and/or by one or more group(s) comprising at least one heteroatom or at least one group comprising at least one heteroatom, the protective group of a hydroxyl function including no hydroxyl, primary amine, secondary amine, phosphate or thiol groups, the hydrocarbon radicals optionally including one cycle or several cycles, optionally one or more heterocycle(s) comprising one or more heteroatom(s), such as oxygen, nitrogen or sulphur atoms, said cycles being optionally condensed. Protective groups of a hydroxyl function that can be used according to the invention are ethers or esters of alkyl, allyl, propargyl, cycloalkyl, aryl (such as benzyl, naphthyl, etc.) or heteroaryl (such as pyridine, indole, benzothiophene, benzofuran, etc.) or polyethylene glycol, esters derived from cholesterol and orthoformate. Particular examples of such groups are the ether or ester groups of tert-butyl, vinyl, benzyl, methoxymethyl, tetrahydropyranyl, triisopropylsilyl, tert-butyldimethylsilyl, tert-butyldiphenyl silyl, benzoyl, acetyl, pivalate, glyceryl, etc. Protective groups of a hydroxyl function such as acetals and acetonides may alternatively be used according to the invention to simultaneously protect two hydroxyl functions of the nucleoside;
and each of the primary amine groups not involved in a bond to said antioxidant molecule or to a lipidising group is substituted by a protective group of a primary amine function fixed to the nitrogen atom of said primary amine group and selected from among linear, branched and/or cyclic, saturated and/or unsaturated, hydrocarbon radicals, aromatic or non-aromatic, optionally substituted, optionally interrupted by one or more heteroatom(s), such as oxygen, nitrogen and sulphur atoms, and/or by one or more group(s) comprising at least one heteroatom or at least one group comprising at least one heteroatom, the protective group of a primary amine function including no hydroxyl, primary amine, secondary amine, phosphate or thiol groups, the hydrocarbon radicals optionally including one cycle or several cycles, optionally one or more heterocycle(s) comprising one or more heteroatom(s) such as oxygen, nitrogen or sulphur atoms, said cycles being optionally condensed. Protective groups of a primary amine function that can be used according to the invention are alkyls, allyls, propargyls, cycloalkyls, aryls (such as benzyl, naphthyl, etc.), polyethylene glycols, alkyl or aryl amides, acetamides (such as benzeneacetamide, pyridine acetamide, etc.), carbamates, azides, triazoles. Particular examples of such groups are tert-butyl, vinyl, benzyl, methoxymethyl, tetrahydropyranyl, triisopropylsilyl, tert-butyldimethylsilyl, tert-butyldiphenyl silyl, benzoyl, acetyl, pivalate, glyceryl, etc., groups.

Thus, the nucleoside of the active compound according to the invention does not comprise any free hydroxyl group or free primary amine group.

In the present description, by “prodrug”, it is meant, in a manner conventional per se, a pharmaceutically-acceptable derivative of a pharmaceutically-active substance, the in vivo transformation of which releases the pharmaceutically-active substance.

By “pharmaceutically-active” molecule, it is meant a molecule for therapeutic use, indicated for preventing and/or treating a disease, and/or for preventing and/or improving one or more of its symptoms.

By “acceptable pharmaceutical” derivative, it is meant any derivative comprising, conjugated with the pharmaceutically-active molecule, a substance that does not cause any adverse, allergic or other undesirable reaction effect when administered to a subject, in particular to a mammal, and in particular to a human, in a form conjugated with the pharmaceutically-active molecule.

Furthermore, in the present description, by “pharmaceutically-acceptable salt”, it should be understood any salt of the active compound implementing, as a counter-ion, a species that does not produce any adverse, allergic or other undesirable reaction effect when administered to a subject, in particular to a mammal. Any non-toxic salt conventionally used in the pharmaceutical field may be implemented in the context of the invention, whether it is formed from organic acids or from inorganic acids, for example a chloride, a bromide, a formate, an acetate, etc. This salt may be synthesised from the active compound and the corresponding acid, according to any conventional chemical method.

Preferably, the salt of the active compound is selected so as to be soluble in oily phases.

In the present description, the expression “lipidated nucleoside” refers to a nucleoside modified by at least one lipidising group in accordance with the invention, such a group conferring a lipidic nature on the nucleoside.

As indicated hereinbefore, the conjugate formed by the antioxidant molecule and the nucleoside carrying a lipidising group in accordance with the invention has a particularly good capacity of absorption in the biological membranes.

Furthermore, the presence of a bond comprising a carboxylic ester group, between the active antioxidant molecule and the lipidated nucleoside, advantageously enables an in situ release of the antioxidant molecule in its active form in all target sites of the organism, by cleavage of the carboxylic ester bond by hydrolysis by esterases, ubiquitous enzymes present in all cells of the organism.

In its form of the active compound, or of a salt of this active compound soluble in oily phases, the prodrug according to the invention is advantageously soluble in lipophilic vehicles, which facilitates the integration thereof within numerous galenic forms, and in particular in emulsions, and in particular nanoemulsions, comprising a lipophilic phase and an aqueous phase, one of these phases being dispersed in the other phase in the form of fine droplets.

The nucleoside included in the active compound according to the invention may carry one or more lipidising group(s), which are covalently fixed thereto by a covalent bond. In the case of a plurality of lipidising groups, these may be identical to one another, or different from one another.

In particular embodiments of the invention, at least one lipidising group, where appropriate each lipidising group, is selected from among:
the groups —CH₂—R′, —CO—CH₂—R′ and —CO—(CH₂)₂—CO—O—R′, where R′ represents cholesterol, tocopherol, tocotrienol, a phenyl ring optionally substituted by one or more group(s) R₅, a naphthyl ring possibly substituted by one or more group(s) R₅, a pyridine ring optionally substituted by one or more group(s) R₅, where R₅ represents a linear or branched, saturated or unsaturated, C1-C25, preferably C4-C25 and preferentially C10-C25, hydrocarbon chain,
an allyl radical or a propargyl radical,
the groups —R and —CO—R, where R represents: a linear or branched, saturated or unsaturated, C1-C25, preferably C4-C25 and preferentially C10-C25, hydrocarbon chain; a group —(CH₂)₂—O—R₅—O—CH₃ where R₅ represents a linear or branched, saturated or unsaturated, C1-C25, preferably C4-C25 and preferentially C10-C25, hydrocarbon chain; a group —CH₂—CH(O—R₆)—O—R₇ where R₆ and R₇, identical or different, each represent a linear or branched, saturated or unsaturated, C1-C25, preferably C4-C25 and preferentially C10-C25, hydrocarbon chain; a group —CH(O—R₆)—O—R₇ where R₆ and R₇, identical or different, each represent a linear or branched, saturated or unsaturated, C1-C25, preferably C4-C25 and preferentially C10-C25, hydrocarbon chain; or a group —CH₂—CH(O—CO—R₆)—O—CO—R₇ where R₆ and R₇, identical or different, each represent a linear or branched, saturated or unsaturated, C1-C25, preferably C4-C25 and preferentially C10-C25, hydrocarbon chain,
the groups —CH₂—R₈ where R₈ represents a triazole group, a benzothiophene group, a benzofuran group or an indole group (benzopyrrole), R₈ being optionally substituted by one or more group(s) R₅ where R₅ represents a linear or branched, saturated or unsaturated, C1-C25, preferably C4-C25 and preferentially C10-C25, hydrocarbon chain,
the groups —(CH₂)₂—O—(CH₂)₂—O—(CH₂)ₚ—O—CH₂—R₁₅, where p is an integer comprised between 2 and 25 and R₁₅ represents a hydrogen atom or a phenyl group;
the groups —CO—CH₂—O—(CH₂)_{q}—O—CH₂—R₁₆, where q is an integer comprised between 2 and 25 and R₁₆ represents a hydrogen atom or a phenyl group;
a triazole group, optionally substituted by a linear or branched, saturated or unsaturated, C1-C25, preferably C4-C25 and preferentially C10-C25, hydrocarbon chain, for example by a geranyl or farnesyl radical.

Otherwise, the lipidising group may form an acetal group with two distinct oxygen atoms of the ribose unit of the nucleoside, this acetal group comprising one or more linear, branched and/or cyclic, saturated or unsaturated, C1-C25 hydrocarbon chain(s), aromatic or not, optionally substituted, optionally interrupted by one or more heteroatom(s) and/or by one or more group(s) comprising at least one heteroatom or at least one group comprising at least one heteroatom, excluding hydroxyl, primary amine, secondary amine, phosphate and thiol groups, and optionally comprising one cycle or several cycles, optionally one or more heterocycle(s) comprising one or more heteroatom(s), the cycles being optionally condensed.

For example, this acetal group may have one of the following general formulas:
—O—CH(—O—)—O—R₅, where R₅ represents a linear or branched, saturated or unsaturated, C1-C25, preferably C4-C25 and preferentially C10-C25, hydrocarbon chain;
O—C(—O—)—R₁₇, where R₁₇ forms with the carbon atom to which it is bonded a cycloalkyl comprising 3 to 25 carbon atoms;
O—C(—O—)(—R₆)—R₇, where R₆ and R₅, identical or different, each represent a linear or branched, saturated or unsaturated, C1-C25, preferably C4-C25 and preferentially C10-C25, hydrocarbon chain.

Such lipidising groups, associated with the absence of free hydroxyl groups and free primary amine groups within the nucleoside, ensure a particularly good solubility of the prodrug according to the invention in oily vehicles.

Examples of lipidising groups that can be used in the context of the invention correspond to the respective general formulas (IVa) to (IVq):

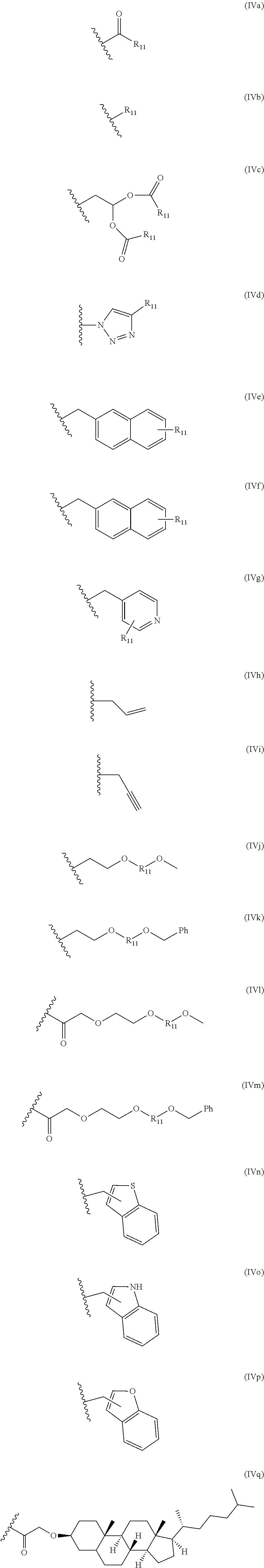

wherein R₁₁ represents a linear or branched, saturated or unsaturated, C1-C25, preferably C4-C25 and preferentially C10-C25, hydrocarbon chain.

A nucleoside is defined in the present description in a manner conventional per se, as an entity having the general formula (V):

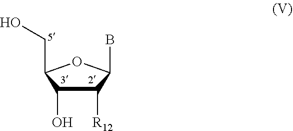

wherein B represents a nucleic base, also called nucleobase or nitrogenous base, and R₁₂ represents a hydroxyl group or a hydrogen atom.

Preferably, the nucleoside according to the invention is a deoxyribonucleoside, wherein, in the formula hereinabove, R₁₂ represents a hydrogen atom.

According to the invention, the nucleoside conjugated to the antioxidant molecule is modified by covalently linking thereto at least one lipidising group in accordance with the invention and by protection of each of its possible hydroxyl groups remaining free by a protective group of a hydroxyl function, as defined hereinabove, as well as of each of its possible primary amine groups remaining free by a protective group of a primary amine function, as defined hereinabove. The nucleoside thus modified by the lipidising group, referred to in the present description as lipidated nucleoside, or nucleolipid, is advantageously non-toxic, in particular for mammals, and in particular for humans, and biodegradable.

The nucleic base included in the nucleoside according to the invention may be:
a natural nitrogenous base, of the purine or pyrimidine type, optionally substituted, in particular by a halogen atom, for example a bromine or iodine atom, or by a linear, branched and/or cyclic, saturated and/or unsaturated, hydrocarbon radical, aromatic or not, optionally substituted, optionally interrupted by one or more heteroatom(s) and/or by one or more group(s) comprising at least one heteroatom or at least one group comprising at least one heteroatom, excluding hydroxyl, primary amine, secondary amine, phosphate or thiol groups, and optionally including a cycle or several cycles, optionally condensed, for example substituted by an ethynyl radical or a triazole ring optionally substituted, for example substituted by a linear or branched, saturated or unsaturated, C1-C25 hydrocarbon chain,
or a non-natural heterocyclic, monocyclic or bicyclic, base, each cycle of which includes from 4 to 7 members,
this nucleic base being, where appropriate, modified so as not to include a free primary amine group.

The nucleic base of the nucleoside according to the invention may be selected from among adenine, guanine, uracil, thymine and cytosine, or derivatives thereof, of respective formulas (VIa) to (VIe):

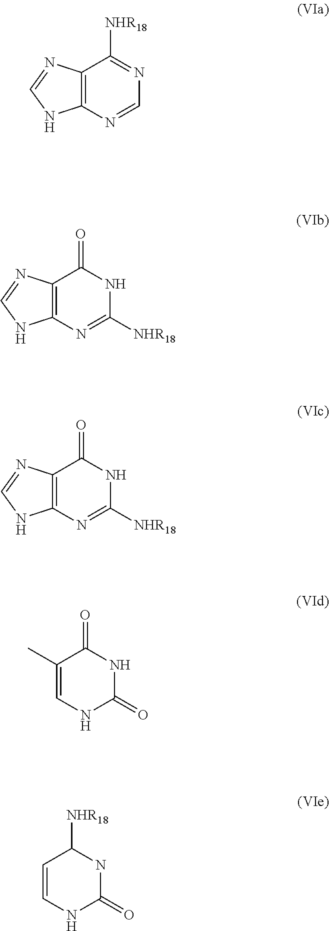

where R₁₈, if it does not represent a lipidising group according to the invention, represents a protective group of a free amine function as defined hereinbefore. Each of these nucleic bases may be modified, at a primary or secondary amine function, by a lipidising group in accordance with the invention.

The bond of the nucleic base to the ribose or deoxyribose unit within the nucleoside according to the invention is formed according to the natural configuration of the nucleosides, i.e. between the carbon atom located in the position 1 of the ribose or deoxyribose unit and the nitrogen atom located in the position 1 for pyrimidines and in the position 9 for purines.

Preferably, the coupling of the antioxidant molecule to the nucleoside is carried out on a first oxygen atom of the nucleoside selected from among the oxygen atom located in the position 5′ of the ribose or deoxyribose unit and the oxygen atom in the position 3′ of the ribose or deoxyribose unit.

Preferably, in such a configuration:
a lipidising group is carried by a second oxygen atom of the nucleoside, different from said first oxygen atom, selected from among the oxygen atom located in the position 5′ of the ribose or deoxyribose unit and the oxygen atom located in the position 3′ of the ribose or deoxyribose unit,
and/or a lipidising group is carried by a nitrogen atom of a primary or secondary amine group of the nucleic base of the nucleoside.

In particular embodiments of the invention, the nucleoside has the general formula (I):

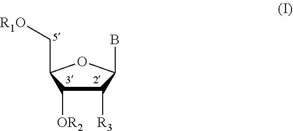

wherein
B represents said nucleic base, optionally substituted at an amine group by a lipidising group, each of the possible primary amine groups of this nucleic base not involved in a bond to the antioxidant molecule or to a lipidising group being substituted by a protective group of a primary amine function as defined hereinbefore, and
either R₁ represents the bond to the antioxidant molecule, and
R₂ represents said lipidising group, and
R₃ represents H or OR₄ where R₄ represents said lipidising group or a protective group of a hydroxyl function as defined hereinbefore,
or R₂ and R₄ form together, with the oxygen atoms to which they are attached, an acetal group comprising one or more linear, branched and/or cyclic, saturated or unsaturated, C1-C25 hydrocarbon chains, aromatic or non-aromatic, optionally substituted, optionally interrupted by one or more heteroatom(s) and/or by one or more group(s) comprising at least one heteroatom or at least one group comprising at least one heteroatom, excluding hydroxyl, primary amine, secondary amine, phosphate and thiol groups, and optionally including one cycle or several cycles, optionally one or more heterocycle(s) comprising one or more heteroatom(s), the cycles being optionally condensed,
or R₁ represents the bond to said antioxidant molecule, and
R₂ represents a lipidising group or a protective group of a hydroxyl function as defined hereinbefore,
R₃ represents OR₄ where R₄ represents a lipidising group,
or R₂ represents the bond to the antioxidant molecule, and
R₁ represents a lipidising group,
R₃ represents H or OR₄ where R₄ represents a lipidising group or a protective group of a hydroxyl function as defined hereinbefore,
or R₂ represents the bond to said antioxidant molecule, and
R₁ represents a lipidising group or a protective group of a hydroxyl function as defined hereinbefore,
R₃ represents OR₄ where R₄ represents a lipidising group.

In embodiments wherein R₁ represents the bond to the antioxidant molecule and R₂ and R₄ form together, with the oxygen atoms to which they are attached, an acetal group, the latter preferably has the formula (VII):

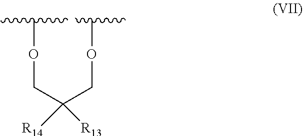

wherein R₁₃ and R₁₄, identical or different, may each represent a hydrogen atom, a C1-C25, for example C1-C6, alkyl group, for example a methyl group, or a C1-C25, for example a C1-C6, oxyalkyl group,
or R₁₃ and R₁₄ may form together, with the carbon atom to which they are attached, a C3-C25, for example C3-C6, cycloalkyl group.

In particular embodiments of the invention, the antioxidant molecule is covalently coupled to the nucleoside via a spacer arm. This spacer arm may contain at least one carboxylic ester group. It may also or alternatively contain at least one carbonyl group bonded to the oxygen atom in the position 5′ of the ribose or deoxyribose unit or to the oxygen atom in the position 3′ of the ribose or deoxyribose unit of the nucleoside or to an oxygen atom of the antioxidant molecule, so as to form a carboxylic ester group with this oxygen atom. Preferably, the spacer arm is devoid of any bond of the phosphoramidate or phosphodiester type, as well as of any disulphide bond.

Preferably, the bond of the antioxidant molecule to the nucleoside, via the spacer arm, includes one or two carboxylic ester group(s).

Preferably, the antioxidant molecule is covalently coupled to the nucleoside via a spacer arm of the general formula (II):

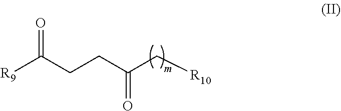

wherein R₉ represents the antioxidant molecule and R₁₀ represents the nucleoside, and
m is equal to 0 and the bond of the antioxidant molecule to the spacer arm is carried by an oxygen atom of the antioxidant molecule and/or the bond of the nucleoside to the spacer arm is carried by an oxygen atom of the nucleoside,
or m is equal to 1 and the bond of the antioxidant molecule to the spacer arm is carried by an oxygen atom of the antioxidant molecule.

Preferably, the pharmaceutically-active antioxidant molecule of the prodrug according to the invention is selected from among α-tocopherol, β-tocopherol, γ-tocopherol, α-tocotrienol, β-tocotrienol, γ-tocotrienol, δ-tocotrienol, fenchol, resveratrol, lipoic acid, retinol, retinal, retinoic acid and ascorbic acid.

In embodiments of the invention, the active compound has the general formula (III):

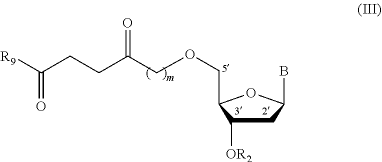

wherein
B represents the nucleic base of the nucleoside, each of the possible primary amine groups of this nucleic base not involved in a bond to said antioxidant molecule or to a lipidising group being substituted by a protective group of a primary amine function as defined hereinbefore, for example by a benzyl radical, the nucleic base being in particular thymine,
R₂ represents the lipidising group, for example a benzyl radical,
R₉ represents the antioxidant molecule, optionally bonded to the spacer arm at one of its oxygen atoms, when it includes one or more oxygen atom(s),
and m is equal to 0 or 1, m being necessarily equal to 0 if the bond of the antioxidant molecule to the spacer arm is not carried by an oxygen atom of the antioxidant molecule.

In other embodiments of the invention, the active compound has the general formula (III′):

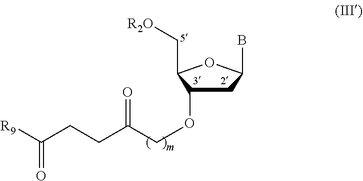

wherein
B represents the nucleic base of the nucleoside, each of the possible primary amine groups of this nucleic base not involved in a bond to said antioxidant molecule or to a lipidising group being substituted by a protective group of a primary amine function as defined hereinbefore, for example by a benzyl radical, the nucleic base being in particular thymine,
R₂ represents the lipidising group, for example a benzyl radical,
R₉ represents the antioxidant molecule, optionally bonded to the spacer arm at one of its oxygen atoms, when it includes one or more oxygen atom(s),
and m is equal to 0 or 1, m being necessarily equal to 0 if the bond of the antioxidant molecule to the spacer arm is not carried by an oxygen atom of the antioxidant molecule.

Active compounds of a particular interest in accordance with the invention have the following general formulas:

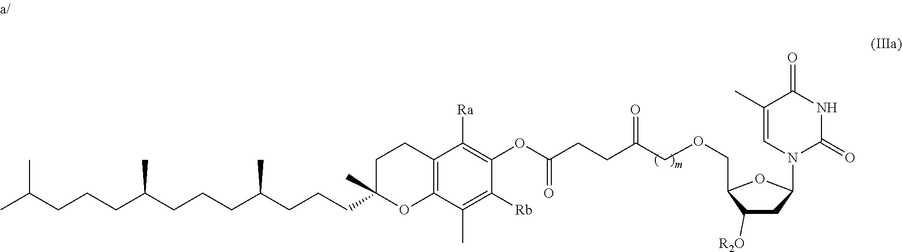

wherein
m is equal to 0 or 1,
Ra and Rb, identical or different, each represent a hydrogen atom or a methyl group,
and R₂ represents the lipidising group, for example a benzyl radical or a group —CO—R₁₁ where R₁₁ represents a linear or branched, saturated or unsaturated, C1-C25, preferably C4-C25 and preferentially C10-C25, hydrocarbon chain,
the antioxidant molecule and the nucleoside in this active compound being respectively a tocopherol isomer and thymidine;

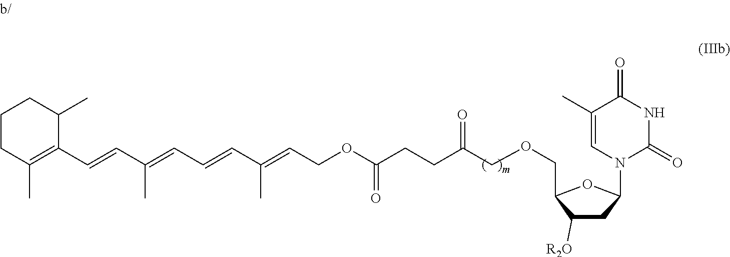

wherein
m is equal to 0 or 1,
and R₂ represents the lipidising group, for example a benzyl radical or a group —CO—R₁₁ where R₁₁ represents a linear or branched, saturated or unsaturated, C1-C25, preferably C4-C25 and preferentially C10-C25, hydrocarbon chain,
the antioxidant molecule and the nucleoside in this active compound being respectively retinol and thymidine;

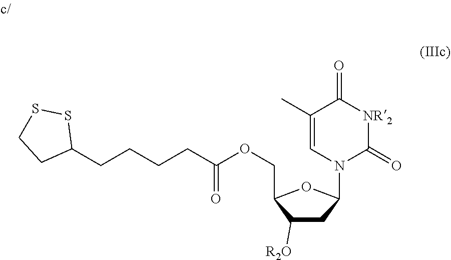

wherein R₂ and R′₂, identical or different, each represent a lipidising group, for example a benzyl radical or a group —CO—R₁₁ where R₁₁ represents a linear or branched, saturated or unsaturated, C1-C25, preferably C4-C25 and preferentially C10-C25, hydrocarbon chain,
the antioxidant molecule and the nucleoside in this active compound being respectively lipoic acid and thymidine;

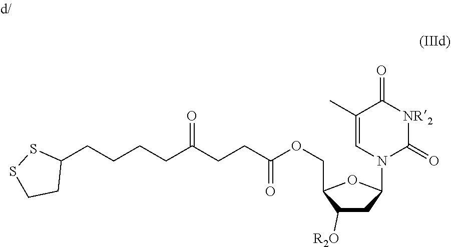

wherein R₂ and R′₂, identical or different, each represent a lipidising group, for example a benzyl radical or a group —CO—R₁₁ where R₁₁ represents a linear or branched, saturated or unsaturated, C1-C25, preferably C4-C25 and preferentially C10-C25, hydrocarbon chain,
the antioxidant molecule and the nucleoside in this active compound being respectively lipoic acid and thymidine;

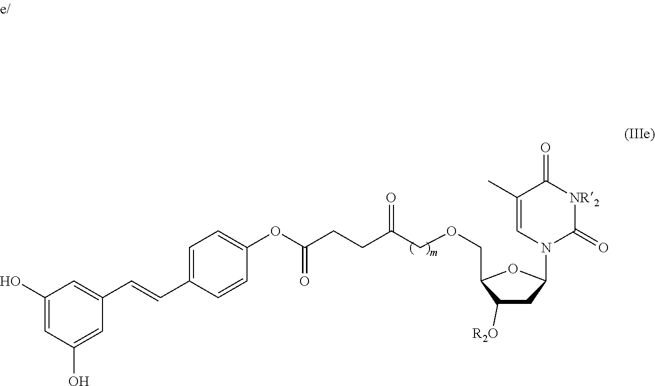

wherein
m is equal to 0 or 1,
and R₂ and R′₂, identical or different, each represent a lipidising group, for example a benzyl radical or a group —CO—R₁₁ where R₁₁ represents a linear or branched, saturated or unsaturated, C1-C25, preferably C4-C25 and preferentially C10-C25, hydrocarbon chain,
the antioxidant molecule and the nucleoside in this active compound being respectively resveratrol and thymidine;

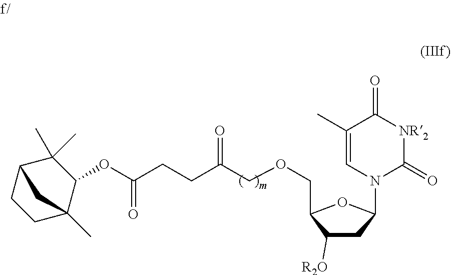

wherein
m is equal to 0 or 1,
and R₂ represents a lipidising group, for example a benzyl radical or a group —CO—R₁₁ where R₁₁ represents a linear or branched, saturated or unsaturated, C1-C25, preferably C4-C25 and preferentially C10-C25, hydrocarbon chain,
the antioxidant molecule and the nucleoside in this active compound being respectively fenchol and thymidine.

In particular, an effect of activating the free fatty acid receptor 2 by the natural antioxidant molecule that is fenchol has been demonstrated by the prior art, in particular illustrated by the publication by Razazan et al., Frontier in Aging Neuroscience, 2021, 13, Article 735933, suggesting a role of the latter on the progression of Alzheimer's disease.

The prodrug according to the invention may be prepared by any chemical synthesis method conventional per se.

In particular, it may be prepared by a method comprising a step of reacting a derivative of the lipidated nucleoside functionalised so as to have a free aldehyde function and a derivative of the antioxidant molecule functionalised so as to have a carbonylvinyl function, according to the Stetter reaction, resulting in the formation of carbon-carbon bonds by 1,4 addition. This addition reaction may be catalysed by a heterocyclic N carbene, for example by the azolium salt of 3-ethyl-5-(2-hydroxyethyl)-4-methylthiazolium bromide. In particular, it may be carried out according to one or several, preferably all, of the following characteristics:
in the presence of a base, such as, for example, 1,8-diazabicyclo[5.4.0]undec-7-ene (DBU),
in an aprotic polar solvent, such as tetrahydrofuran, or dimethylformamide, or in a protic polar solvent such as water,
at a temperature between 40 and 120° C., for example about 75° C.
and/or for 12 to 60 hours, for example about 48 hours.

The lipidated nucleoside, its derivative with a free aldehyde function, and the derivative of the antioxidant molecule with a carbonylvinyl function may be prepared in any manner conventional per se.

Alternatively, the prodrug according to the invention may be prepared by a synthesis method comprising a step of esterifying a derivative of the lipidated nucleoside functionalised so as to have a free carboxylic acid function by the antioxidant molecule having a free hydroxyl function, this esterification reaction being carried out in any manner conventional per se.

Another aspect of the invention relates to a pharmaceutical composition containing, as an active ingredient, a prodrug according to the invention in a pharmaceutically-acceptable vehicle.

In the present description, by “pharmaceutically-acceptable vehicle”, it is meant any vehicle useful for the preparation of a pharmaceutical composition and which is generally safe, non-toxic and neither biologically nor otherwise undesirable for the subject to be treated, in particular for mammals and in particular humans. The pharmaceutical composition according to the invention may be formulated in any dosage form, in particular in a form suitable for oral, parenteral, intranasal, rectal, pulmonary or topical administration.

Preferably, it is in a form suitable for administration by topical application, in particular on the skin and/or mucous membranes, for parenteral administration, in particular by intravenous injection, or for oral administration.

Preferably, the pharmaceutical composition according to the invention is in the form of an emulsion comprising a lipophilic phase and an aqueous phase, the prodrug being contained in the lipophilic phase. The prodrug then consists of said active compound or one of its oil-soluble salts.

Preferably, it consists of a nanoemulsion, or submicronic emulsion, i.e. an emulsion wherein the average size of the globules of the phase dispersed in the other phase is smaller than 300 nm, in particular comprised between 10 and 250 nm.

Where appropriate, it may be gelled, by incorporation of one or more gelling agent(s), conventional per se, for example selected from among branched copolymers of ethylene oxide and polypropylene (poloxamers), carbomers, cellulose derivatives such as hydroxyethyl cellulose, ethylcellulose and methylcellulose, etc. It then consists of a gelled emulsion, preferably a gelled nanoemulsion, which can in the latter case be referred to as “nano-emulgel”. Preferably, the gelling agent(s) are contained in the aqueous phase of the composition.

Preferably, the composition according to the invention is in the form of an oil-in-water emulsion, which in particular has the advantages of better absorption in the organism, and better tolerance by treated subjects when administered intravenously.

This does not exclude the forms of water-in-oil emulsion, or multiple emulsions, for example water-in-oil-in-water, which also fall within the scope of the invention, or else the SEDDS (Self-Emulsifying Drug Delivery System) forms.

In particular embodiments of the invention, the composition comprises, by weight with respect to the total weight of the composition, 1 to 35% of the lipophilic phase, preferably 10 to 30% of the lipophilic phase, for example about 20% of the lipophilic phase.

Of course, all of the constituents of the composition according to the invention are selected so as to be biocompatible, i.e. compatible with an administration to an individual, in particular a mammal and more particularly a human.

The lipophilic phase may comprise any biocompatible oil conventional per se in the field of the galenic formulation, or a mixture of such oils. For example, it may comprise one or more of the following ingredients: fatty acids and fatty acid esters comprising carboxylic acids of different chain lengths, saturated or unsaturated, like arachidic acid, capric acid, caprylic acid, caproic acid, myristic acid, palmitic acid, stearic acid, lauric acid, oleic acid, linoleic acid, linolenic acid or docosahexaenoic acid, fatty acids esterified with glycerol forming mono-, di- or triglycerides, which may be synthetic or originate from natural sources like, for example, soybean oil, olive oil, sesame oil, cottonseed oil, castor oil, poppyseed oil, copra oil, coconut oil, linseed oil, sunflower oil, C6-C12 saturated fatty acids triglycerides oil, and fish oils, the products commercialised under the names Capmul® MCM, Captex® 300, Miglyol® 812, glyceryl monooleate, triacetin, acetylated monoglycerides, tristearin, glyceryl behenate, and diacetyl tartaric acid and monoglycerides esters, etc.

Preferably, the pharmaceutical composition according to the invention further contains an emulsifying agent, which may be of any type conventional per se, in particular of the ionic or non-ionic type, or a mixture of such emulsifying agents. For example, the emulsifying agent(s) may be selected from among:
phospholipid surface agents of natural or synthetic origin such as egg or soy lecithins, pegylated phospholipids;
the other fatty esters, such as myristyl myristate, isopropyl palmitate, benzyl benzoate, sorbitan esters (SPAN) such as laurate, palmitate, oleate stearate and sorbitan trioleate;
polyhydroxyethylene sorbitan esters or polysorbates, like polysorbates 20, 60, 80 or sucroesters;
glycerides conjugated to other species, like polyethylene glycol, for example the products commercialised under the names Labrasol®, Labrafac®, Cremophor EL®;
fatty alcohols such as stearic, lauric or benzyl alcohol;
or any of mixtures thereof.

Preferably, the total concentration of emulsifying agent(s) in the composition is comprised between 0.5 and 15% by weight, preferably between 0.5 and 10% by weight, preferentially between 1 and 7.5% by weight, and more preferentially between 1 and 5% by weight, with respect to the total weight of the composition. As an example, when the emulsifying agent is a lecithin, such as egg lecithin E80, the concentration of this emulsifying agent in the composition is preferably comprised between 1 and 2.5% by weight, for example about 1.5% by weight. When the emulsifying agent is a polysorbate, such as polysorbate 80, the concentration of this emulsifying agent in the composition is preferably comprised between 1 and 5% by weight, for example about 2.5% by weight.

The pharmaceutical composition according to the invention, in the form of an emulsion, in particular an oil-in-water emulsion, and in particular a nanoemulsion, advantageously forms a system that is particularly effective for the delivery of the prodrug according to the invention. It has been discovered by the present Inventors that it promotes even more the passage of the antioxidant molecule through the biological membranes.

This system also allows protecting the prodrug from digestive enzyme hydrolysis before absorption in the biological membranes, which is highly advantageous for oral administration.

Formulated within such a delivery system, the antioxidant molecule included in the prodrug according to the invention benefits from both the absorption promoter nature of the lipidated nucleoside according to the invention, and that of the lipidic emulsion system, in particular of the lipidic nanoemulsion. In particular, this makes it possible for it to cross the blood-brain barrier, which makes it particularly suitable for the treatment of brain diseases.

It is furthermore released in a doubly delayed manner in the organism, after release out of the lipophilic phase globules, then activation by separation from the lipidated nucleoside, by cleavage of the carboxylic ester bond by the esterases present ubiquitously in the organism.

The pharmaceutical composition according to the invention may comprise, expressed in % by weight with respect to the total weight of the composition, an amount comprised between 0.1% and 10%, preferably between 0.1% and 5%, preferentially between 1 and 5%, or between 2 and 5%, and for example about 2% to 3%, of the prodrug according to the invention.

The concentration of the prodrug according to the invention in the lipophilic phase of the composition, when the prodrug consists of said active compound or one of its oil-soluble salts, is preferably comprised between 0.01 and 50%, preferably between 0.1% and 30%, preferentially between 0.1% and 20%, and even more preferentially between 0.1% and 10%, by weight with respect to the total weight of the lipophilic phase.

Pharmaceutical compositions that are particularly preferred according to the invention, in the form of an emulsion comprising a lipophilic phase and an aqueous phase, meet one or more of the following features, according to any possible combination of these features, and in particular all of these features:
it consists of a nanoemulsion, preferably of the oil-in-water type,
it comprises from 0.1 to 10% by weight, in particular from 2 to 5% by weight, and for example 3% by weight, with respect to the total weight of the composition, of the composition, of the prodrug according to the invention,
the prodrug is solubilised in a lipophilic vehicle, comprising for example, or consisting of, the product known under the name Miglyol® 812,
the lipophilic phase represents 10 to 30%, by weight, for example 20% by weight, of the total weight of the composition,
it contains at least one emulsifying agent selected from among egg lecithins and polysorbate 80, or any of mixtures thereof,
and/or the amount of emulsifying agent(s) in the composition is comprised between 1 and 7.5% by weight, preferably between 2 and 7.5% by weight, and preferentially between 3 and 5% by weight, with respect to the total weight of the composition.

In all cases, the amount of water in the composition is adjusted according to that of the other constituents, to obtain a total weight amount of the constituents equal to 100%.

The pharmaceutical composition according to the invention, in the form of a nanoemulsion, is characterised in particular by a white-milky appearance and, preferably:
a Gaussian particle-size distribution with an average diameter, measured by dynamic light scattering (DLS) or quasi-elastic light scattering (QELS), smaller than 300 nm, preferably comprised between 10 and 250 nm,
a polydispersity index (PDI) measured by DLS lower than 0.25
and/or a zeta potential, measured by electrophoretic light scattering (ELS) of about −35 mV.

Advantageously, its macroscopic and granulometric characteristics are stable over time, over a duration as long as several weeks.

The pharmaceutical composition according to the invention may comprise, as an active ingredient, only the prodrug according to the invention. It may alternatively contain this prodrug in a mixture with one or more other pharmaceutically-active substance(s), acting, or not, in synergy with the antioxidant molecule of the prodrug according to the invention.

In particular embodiments of the invention, the pharmaceutical composition further contains a pharmaceutically-active antioxidant molecule in free form, i.e. not conjugated to another molecule. This antioxidant molecule in free form may be identical to that included in the prodrug according to the invention, or be different from the latter. It may be present in the pharmaceutical composition according to the invention as such or, where appropriate, in the form of one of its pharmaceutically-acceptable oil-soluble salts.

As an example, the antioxidant molecule in free form may be selected from among α-lipoic acid, fenchol, resveratrol, fat-soluble vitamins like vitamin A, retinol, β-carotene and vitamin E and derivatives thereof, including all tocopherols and tocotrienols and derivatives thereof like vitamin E succinate, vitamin E acetate, and vitamin E polyethylene glycol succinate (TPGS), or any one of their mixtures.

In particular, such an embodiment allows, highly advantageously, modulating the release of the antioxidant molecule in the organism, by combining an immediate release of the antioxidant molecule contained in free form in the composition, and a delayed release of the antioxidant molecule included in the prodrug according to the invention, by the in vivo hydrolysis of the latter, at the carboxylic ester bond, by the esterases present in the organism.

The pharmaceutical composition according to the invention may further contain, in addition to the constituents defined hereinbefore, any pharmaceutically-acceptable excipient or additive, or a mixture of such excipients and/or additives. By “pharmaceutically-acceptable”, it is meant that the excipient or the additive does not cause any adverse, allergic effect or other undesirable reaction when administered to a subject, in particular to a mammal, and particularly to a human. Such an excipient or additive may be a diluent, an adjuvant, in particular lipidic, or any other substance conventional per se for the formulation of medicines, such as a surfactant, a preservative, an isotonising agent, an agent for adjusting the osmolality and/or the pH, in particular sodium hydroxide, glycerol, etc., In particular, the aqueous phase may contain one or more organic co-solvent(s), for example each selected from among ethanol, polyethylene glycol, propylene glycol, glycerol, macrogols or polyoxyethylene glycols, for example PEG 200, PEG 300 or PEG 400.

The pharmaceutical composition according to the invention may further contain one or more targeting ligand(s) such as a pegylated lipid (phospholipid), an antibody, a peptide, an aptamer, etc.

The pharmaceutical composition according to the invention may be packaged in a multidose container, or in the form of unit doses each containing a therapeutically-effective amount of the prodrug according to the invention. Preferably, the concentration of the prodrug in each dose of the pharmaceutical composition according to the invention is selected so as to deliver to the subject, at each administration, an amount of prodrug that is effective to achieve the desired therapeutic response.

The pharmaceutical composition according to the invention may optionally be sterile, preferably at a sterility assurance level (SAL) less than or equal to 10⁻⁶ according to the European Pharmacopoeia 10.0.

The pharmaceutical composition according to the invention may be prepared by any method conventional per se.

When it is in the form of an emulsion, it is preferably prepared by a method comprising a phase inversion step.

As an example, a method for preparing a pharmaceutical composition according to the invention comprises the following successive steps:
solubilising the prodrug according to the invention in a lipophilic phase, said lipophilic phase optionally containing an oil, possibly a lipophilic surface agent and optionally other adjuvants, in particular lipidic, for example α-tocopherol in free form and/or a pegylated lipid;
preparing an aqueous phase comprising at least one emulsifying agent, and optionally one or more osmolality- and pH-adjusting agent(s);
heating, separately, each of these two phases, at a temperature for example comprised between 70 and 80° C.;
mixing by phase inversion by introducing the aqueous phase into the lipophilic phase at very high speed, in particular by means of an Ultra-Turrax® disperser, so as to form a coarse emulsion, the lipidic globules of which have an average diameter larger than 2 μm;
reducing the size of the lipidic globules to obtain a nanoemulsion by means of a high-pressure homogeniser, a microfluidiser or an ultrasound probe, in order to obtain that 100% of the lipidic globules have a size smaller than 1 μm with an average diameter comprised between 140 and 300 nm;
and, where appropriate, the sterilisation of the nanoemulsion thus obtained, for example in an autoclave at 121° C. for 15 minutes, or by sterilising filtration.

Another aspect of the invention relates to the use of a prodrug according to the invention, or of a pharmaceutical composition according to the invention, as a medicine, for the therapeutic treatment of a subject in need thereof, i.e. suffering or likely to suffer from the disease, in particular of a mammal, and particularly of a human.

In the present description, by “treatment”, it is intended to mean a prophylactic and/or curative treatment of a disease, comprising the prevention of the disease or the total or partial prevention of one or more of the symptoms of the disease and/or the total or partial healing of the disease and/or the total or partial disappearance of one or more of its symptoms.

Preferably, the prodrug according to the invention or the composition according to the invention are administered to the subject in a therapeutically-effective amount.

By “therapeutically-effective amount”, it is meant the amount which, when administered to a subject to treat the disease, is sufficient to ensure such a treatment of the disease.

The therapeutically-effective amount of the prodrug according to the invention depends on many factors, such as the particular disease and its severity, the age, the weight, etc., of the subject to be treated, the used antioxidant molecule, the route and the administration form, etc. The therapeutically-effective amount of the prodrug according to the invention will be determined by the physician for each individual case.

The administration of the prodrug or of the pharmaceutical composition according to the invention to the subject to be treated may be carried out by any route conventional per se, in particular parenterally, for example by the subcutaneous, subdural, intravenous, intramuscular, intrathecal, intraperitoneal, intracerebral, intra-arterial or intralesional route; by the intranasal route; by the rectal route; by the pulmonary route, for example by aerosol or inhalation; by the oral route; or topically, for example on the skin or mucous membranes.

The determination of the administration dosage of the prodrug or of the pharmaceutical composition according to the invention falls within the competence of the physician. For example, the prodrug or the pharmaceutical composition may be administered to the subject in need thereof once or twice a day, over a long period, at regular intervals, or in a targeted manner during an acute episode of the disease.

In particular implementations of the invention, the prodrug or the pharmaceutical composition can be used for the treatment of radiological or chemical burns, in particular burns caused by yperite.

Yperite is a combat gas causing serious burns, which has been used in numerous wars, from World War 1 to more recently the Iran-Irak conflict of the 1980s. Massively stored in the arsenals of the cold war, it is still to date one of the most critical chemical war agents as it imposes a very restrictive protection and a heavy medical treatment. The old battlefields remain polluted by unexploded chemical bombshells containing large amounts of yperite, for example on the shores of the North Sea.

The prodrug and the pharmaceutical composition according to the invention find a particularly advantageous application for the treatment of radiological burns or chemical burns, in particular caused by yperite, taking advantage of the antioxidant and healing properties of the pharmaceutically-active molecule of the prodrug according to the invention. In particular, the pharmaceutical composition according to the invention could be advantageously used when disarming the bombshells remaining in old battlefields, and also in the event of a risk of attack in the Middle East.

In such an application field, an administration of the prodrug or the pharmaceutical composition according to the invention by topical, cutaneous or ophthalmic route, proves to be particularly preferred. Configurations of the pharmaceutical composition according to the invention comprising a gelling agent, in particular in the aqueous phase when the composition is in the form of an emulsion, are particularly preferred for cutaneous or ophthalmic topical administrations.

In other particular implementations of the invention, the prodrug or the pharmaceutical composition according to the invention are used for the treatment of a neurodegenerative disease, in particular Parkinson's disease, Alzheimer's disease, or any other disease for which the pharmacological target is the central nervous system.

Many neurodegenerative diseases, such as Parkinson's disease, Alzheimer's disease, etc., remain today incurable and the developed symptomatic treatments have many limits. In particular, a major physiological problem in the development of a chronic treatment is the crossing of the blood-brain barrier (BBB).

It has been discovered by the present inventors that the prodrug according to the invention, and the pharmaceutical composition that contains it, all the more in the form of a lipidic nanoemulsion, advantageously ensure a delivery of the antioxidant molecule of the prodrug within the central nervous system, in neuronal cells and intracellular organelles (lysosomes). More particularly, it has been discovered by the inventors that the combined effect of the lipidated nucleoside conjugated to the antioxidant molecule and of the lipidic nanoemulsion form allows conveying the antioxidant molecule to the central nervous system, the prodrug according to the invention then being capable of penetrating into the human neurons, within which the antioxidant molecule is released by hydrolysis catalysed by the esterases present in situ. In particular, such a targeted delivery advantageously allows delaying the progress of the neurological disease and its symptoms. Mechanisms occurring at the cellular level that are behind such an advantageous result will not assumed here. It is possible that part of it comes from the fact that, in addition to the released antioxidant molecule, the nucleolipid released from the prodrug is then capable of acidifying the lysosomes, resulting in a recovery of the lysosomal function degraded by the disease.

In such a context of application to the treatment of degenerative diseases, the prodrug or the pharmaceutical composition according to the invention are preferably administered to the subject to be treated by a systemic administration route, in particular orally or parenterally, and particularly intravenously.

The prodrug or the pharmaceutical composition according to the invention may alternatively be used for the treatment of ophthalmic diseases, such as AMD, to promote skin healing, or for cosmetic applications, in particular for anti-ageing skin treatment.

The present invention is also expressed in the form of a method of prophylactically or curatively treating a disease in a subject. In particular, the subject may be a mammal and preferably a human. This method comprises administering to said subject in need thereof a therapeutically-effective amount of the prodrug or of the pharmaceutical composition according to the invention. This method may have one or several of the features described hereinabove with reference to the use of the prodrug or of the pharmaceutical composition according to the invention as a medicine.

The present invention is also expressed in the terms of the use, for the prophylactic or curative treatment of a disease in a subject, or for the manufacture of a medicine, of a prodrug or a pharmaceutical composition according to the invention.

This use may meet one or more of the features described hereinabove with reference to the prodrug and to the pharmaceutical composition according to the invention and to their use.

The prodrug and the pharmaceutical composition according to the invention may also be used for the manufacture of medical devices, in particular for the infusion of prostheses, which advantageously ensures the preservation of the mechanical properties and the performances of these during use thereof.

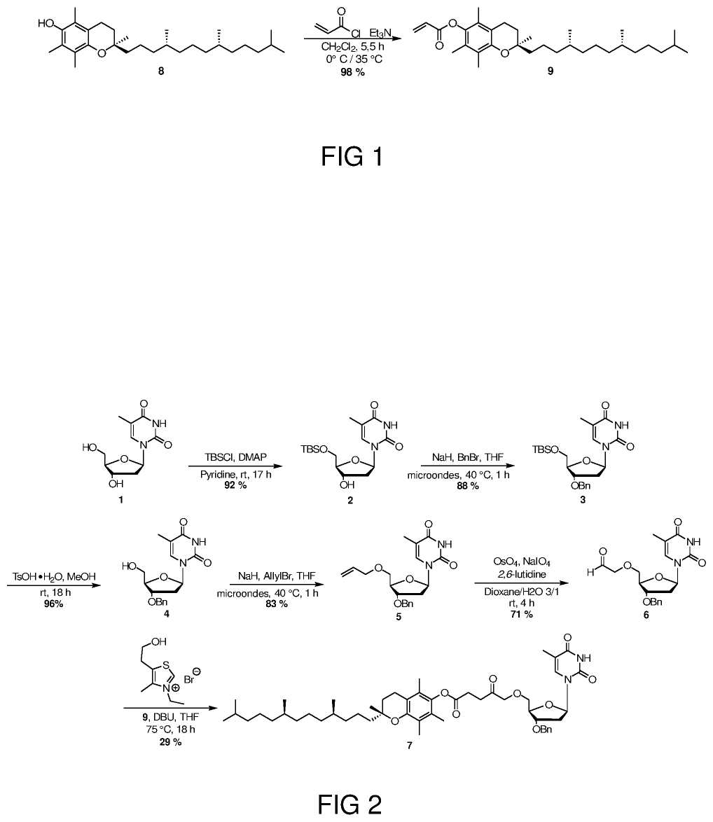

The features and advantages of the invention will appear more clearly in light of the examples of implementation hereinafter, given for mere illustration and non-limiting of the invention, with reference to FIGS. 1 to 12, wherein:

FIG. 1 shows the reaction scheme for the synthesis of an α-tocopherol derivative carrying a carbonylvinyl function.

FIG. 2 shows the synthesis scheme of a prodrug in accordance with the invention comprising an α-tocopherol molecule bonded to the thymidine nucleoside carrying a lipidising group (benzyl group on the oxygen in the position 3′ of the deoxyribose unit).

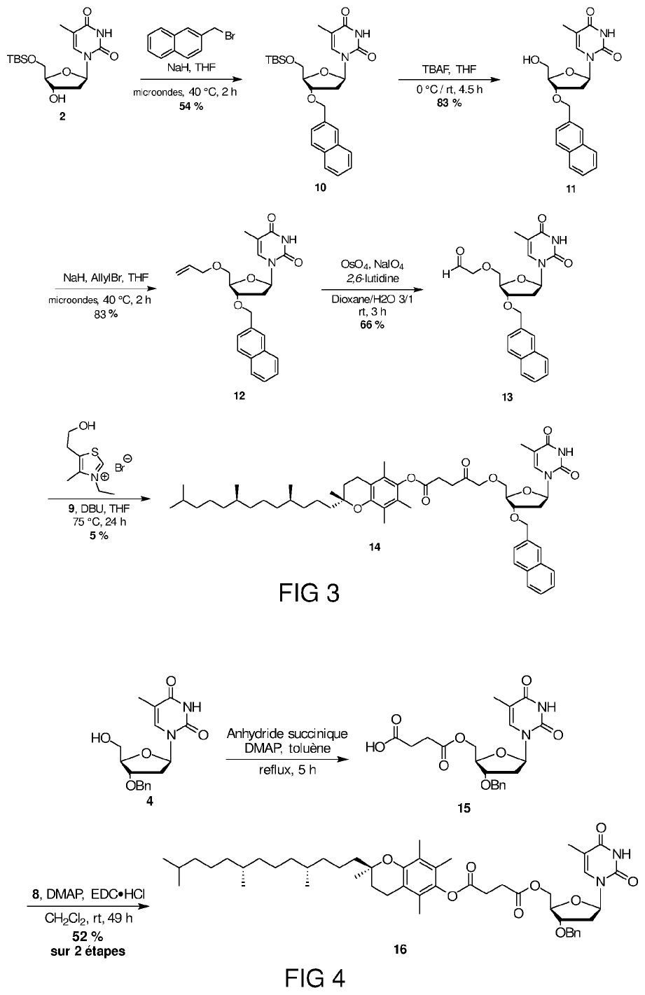

FIG. 3 shows the synthesis scheme of a prodrug in accordance with the invention comprising an α-tocopherol molecule bonded to the thymidine nucleoside carrying a lipidising group (methyl-naphthyl group on the oxygen in the position 3′ of the deoxyribose unit).

FIG. 4 shows the synthesis scheme of a prodrug in accordance with the invention comprising an α-tocopherol molecule bonded to the thymidine nucleoside carrying a lipidising group (benzyl group on the oxygen in the position 3′ of the deoxyribose unit).

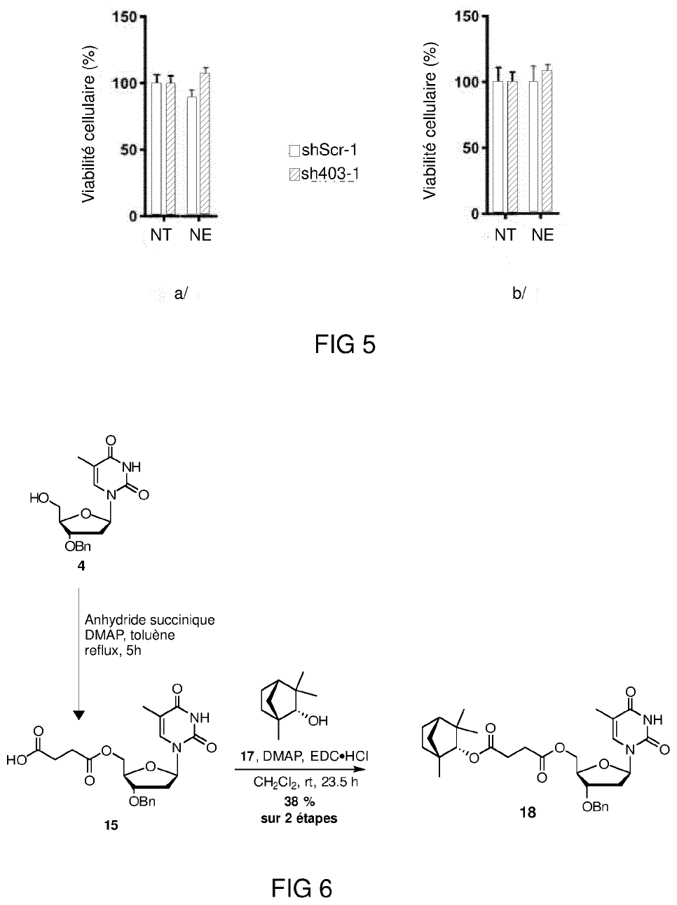

FIG. 5 represents graphs showing the % cell viability of human neuronal cells untreated (NT) or after 24 h (in a/) or 48 h (in b/) of treatment with a pharmaceutical composition in the form of a nanoemulsion in accordance with the invention (NE), containing 2% w/w of active compound.

FIG. 6 shows the synthesis scheme of a prodrug in accordance with the invention comprising a fenchol molecule bonded to the thymidine nucleoside carrying a lipidising group (benzyl group on the oxygen in the position 3′ of the deoxyribose unit).

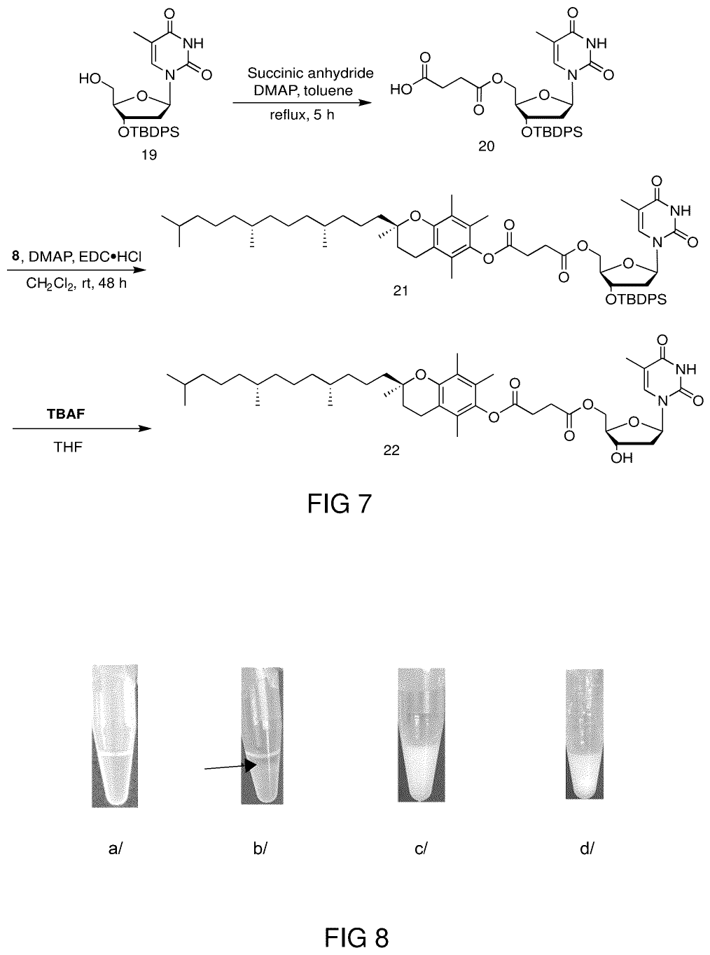

FIG. 7 shows the synthesis scheme of a compound not in accordance with the invention comprising an α-tocopherol molecule bonded to the thymidine nucleoside with no lipidising group; TBDPS represents a tertbutyldiphenylsilyl group.

FIG. 8 shows dilutions in the oily vehicle Miglyol® 812 of: a/a compound in accordance with the invention comprising an α-tocopherol molecule bonded to the thymidine nucleoside carrying a lipidising group (benzyl group on the oxygen in the position 3′ of the deoxyribose unit), diluted to 5% w/w; b/a comparative compound similar but devoid of the lipidising group, diluted to 1% w/w; c/the same comparative compound diluted to 2% w/w; d/the same comparative compound diluted to 5% w/w.

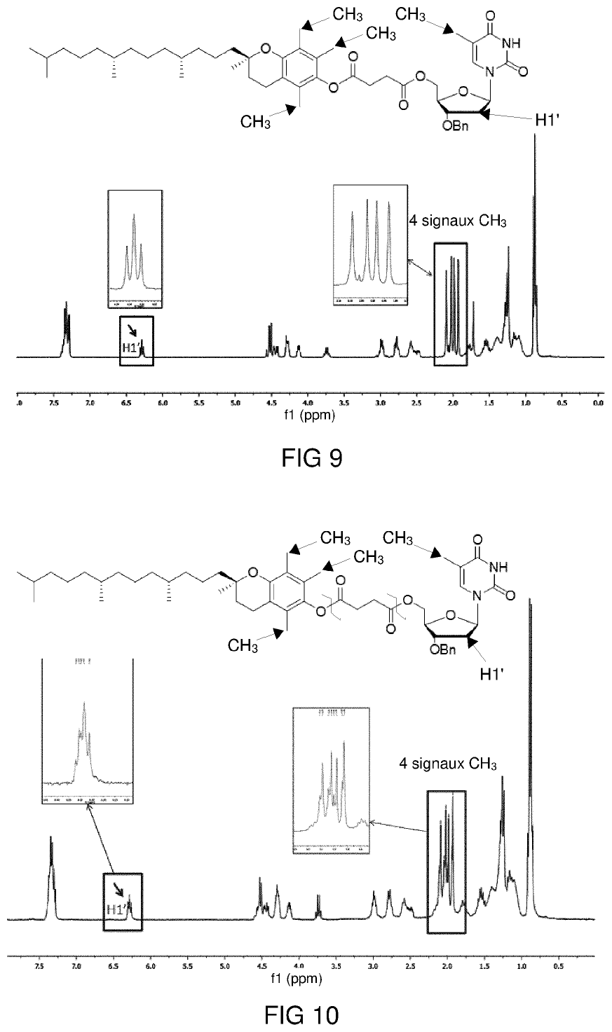

FIG. 9 shows the NMR ¹H spectrum of a compound in accordance with the invention comprising an α-tocopherol molecule bonded to the thymidine nucleoside carrying a lipidising group (benzyl group on the oxygen in the position 3′ of the deoxyribose unit).

FIG. 10 shows the NMR ¹H spectrum of the compound of FIG. 9 after 8 h of UV irradiation.

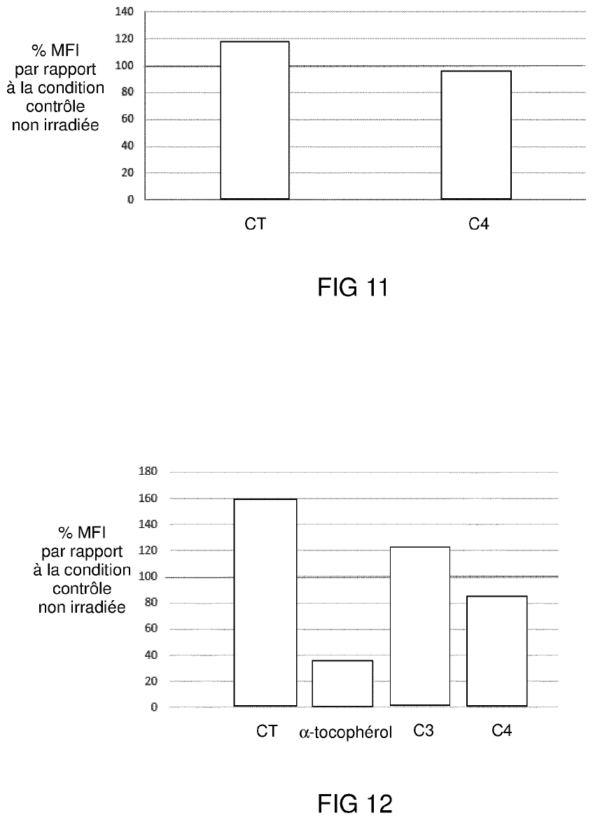

FIG. 11 shows a bar graph representing the % of mean fluorescence intensity (MFI) with respect to a non-irradiated control condition, obtained in the presence of the oxidative stress indicator CM-H₂DCFDA, for keratinocytes irradiated according to the solar spectrum after incubation for 30 min in the presence of a composition in accordance with the invention in the form of a nanoemulsion containing 5% w/w of an active compound according to the invention, diluted to 1/1,000^{th} in the medium (“C4”) or in the absence of such a composition (“CT”).

FIG. 12 shows a bar graph representing the % of mean fluorescence intensity (MFI) with respect to a non-irradiated control condition, obtained in the presence of the oxidative stress indicator CM-H₂DCFDA, for keratinocytes irradiated according to the solar spectrum after incubation for 48 h in the presence of: a composition in accordance with the invention in the form of a nanoemulsion containing 5% w/w of an active compound according to the invention, diluted to 1/1,000^{th} in the medium (“C4”); a composition in accordance with the invention in the form of a nanoemulsion containing 3% w/w of an active compound according to the invention, diluted to 1/1,000^{th} in the medium (“C3”); α-tocopherol at 0.5% w/v; or in the absence of any compound (“CT”).

### EXAMPLE 1

A α-tocopherol derivative (compound 9) is obtained from α-tocopherol 8 according to the reaction scheme shown in FIG. 1.

To this end, triethylamine Et₃N (1.2 eq., 100 μL, 0.70 mmol) is added in drops to a solution of α-tocopherol 8 (1.0 eq., 250 mg, 0.58 mmol) in dichloromethane (0.1 M, 5.8 mL). At 0° C., acryloyl chloride (2.0 eq., 94 μL, 1.16 mmol) is added in drops. The reaction medium is stirred for 2 h at 0° C. before being stirred for 3.5 h at 35° C. After cooling to room temperature, the medium is washed 3 times with a saturated NaHCO₃ solution. The organic phase is recovered, dried over Na₂SO₄, filtered and concentrated under reduced pressure. The reaction crude is purified by silica gel chromatography (EtOAc/pentane 1/99) to obtain the compound 9 (274.5 mg, 98%, colourless oil).

The characterisation data of this compound are in compliance with the data published in the literature (Xie et al., J. Am. Chem. Soc., 2020, 142, 16787-16794).

### EXAMPLE 2

An active compound according to the invention (compound 7) is prepared according to the reaction scheme shown in FIG. 2, from the α-tocopherol derivative 9 prepared in Example 1, and thymidine 1.

From thymidine 1, the compound 2 is obtained as described in the publication by Maturano et al., Eur. J. Org. Chem., 2012, 721.

In a flask at 0° C., sodium hydride NaH (60% dispersion in an oil, 280 mg, 7.01 mmol) is added in portions in a solution of the compound 2 (1 eq., 1.00 g, 2.80 mmol) in tetrahydrofuran THF (0.1 g/mL, 10 mL). The reaction medium is activated by microwaves (CEM machine, 200 W) at 40° ° C. for 4.5 min. Benzyl bromide (2.5 eq., 833 μL, 7.01 mmol) is added to the reaction medium which is subjected afterwards to a second activation with microwaves (CEM machine, 200) at 40° C. for 1 h. After cooling to room temperature, the reaction is stopped with a saturated solution of ammonium chloride NH₄Cl. The medium is extracted 3 times with dichloromethane. The organic phases are combined, dried over Na₂SO₄, filtered and concentrated under reduced pressure. The reaction crude is purified by silica gel chromatography (EtOAc/pentane 30/70) to obtain the compound 3 (1.013 g, 88%, colourless oil), the characterisation data of which are in accordance with those published in the literature (Teste et al., Carbohydrate Research, 2008, 343, 1490).

Paratoluenesulphonic acid monohydrate (0.2 eq., 86 mg, 0.45 mmol) is added to a solution of the compound 3 (1.0 eq., 1.013 g, 2.27 mmol) in methanol (0.1 g/mL, 9 mL). After stirring for 18 h at room temperature, the reaction medium is concentrated under reduced pressure. The residue is dissolved in dichloromethane. The organic phase is washed with a saturated NaHCO₃ solution; then brine. The organic phase is recovered, dried over Na₂SO₄, filtered and concentrated under reduced pressure. The crude oil reaction is purified by chromatography on silica gel (MeOH/CH₂Cl₂ 5/95) to obtain the compound 4 (724 mg, 96%, white foam).

Rf=0.25 (Pentane/EtOAc 30/70)

IR (ATR) vₘₐₓ (cm⁻¹) 3,422, 3,186, 3,062, 3,038, 2,928, 1,683, 1,471, 1,405, 1,366, 1,324, 1,275, 1,203, 1,132, 1,096, 1,058, 960, 910, 787, 736, 699, 651, 606, 560, 492

NMR—¹H (300 MHz, CDCl₃) δ (ppm) 8.99 (s, 1H), 7.42-7.29 (m, 6H), 6.14 (appears t, J=6.6, 7.5 Hz, 1H), 4.54 (AB system, J=11.8 Hz, 2H), 4.33-4.26 (m, 1H), 4.20-4.13 (m, 1H), 3.93 (dd, J=2.4, 11.7 Hz, 1H), 3.75 (dd, J=3.0, 12 Hz, 1H), 2.48-2.28 (m, 2H), 1.90 (s, 3H)

NMR—¹³C (75.5 MHz, CDCl₃) δ (ppm) 163.9, 150.5, 137.6, 137.2, 128.7, 128.1, 127.8, 111.2, 87.5, 85.3, 78.7, 71.8, 62.9, 37.3, 12.6

HRMS (ESI): calculated for C₁₇H₂₀N₂O₅Na [M+Na]⁺ 355.12644; found 355.1265. In a vial at 0° C., NaH (60% dispersion in an oil, 248 mg, 6.20 mmol) is added in portions in a solution of the compound 4 (1 eq., 825 mg, 2.48 mmol) in THF (0.1 g/mL, 8.3 mL). The reaction medium is activated by microwaves (CEM machine, 200 W) at 40° C. for 2 min. the allyl bromide (2.5 eq., 536 μL, 6.20 mmol) is added to the reaction medium which is subjected afterwards to a second activation with microwaves (CEM machine, 200) at 40° C. for 1 h. After cooling to room temperature, the reaction is stopped with a saturated NH₄Cl solution. The medium is extracted 3 times with dichloromethane. The organic phases are combined, dried over Na₂SO₄, filtered and concentrated under reduced pressure. The reaction crude is purified by silica gel chromatography (EtOAc/pentane 40/60 to 50/50) to obtain the compound 5 (766 mg, 83%, colourless oil).

Rf=0.37 (Pentane/EtOAc 50/50)

IR (ATR) vₘₐₓ (cm⁻¹) 3,180, 3,064, 3,035, 2,925, 2,861, 1,682, 1,468, 1,434, 1,402, 1,364, 1,329, 1,274, 1,202, 1,096, 1,055, 1,029, 960, 914, 884, 785, 733, 698, 670, 645, 608, 558, 491

NMR—¹H (300 MHZ, CDCl₃) δ (ppm) 9.28-9.07 (br. s, 1H), 7.63 (s, 1H), 7.40-7.27 (m, 5H), 6.39 (dd, J=6.0, 7.8 Hz, 1H), 5.97-5.82 (m, 1H), 5.32-5.18 (m, 2H), 4.54 (dd, J=12.0, 26.7 Hz, 2H), 4.28-4.21 (m, 2H), 4.06-4.01 (m, 2H), 3.74 (dd, J=2.4, 10.8 Hz, 1H), 3.58 (dd, J=2.1, 10.5 Hz, 1H), 2.47 (ddd, J=1.8, 5.7, 12.9 Hz, 1H), 2.16-2.05 (m, 1H), 1.89 (d, J=0.9 Hz, 3H)

NMR—¹³C-NMR (75.5 MHz, CDCl₃) δ (ppm) 164.0, 150.5, 137.6, 136.0, 134.0, 128.6, 128.0, 127.7, 117.6, 110.9, 85.3, 84.1, 79.3, 77.4, 72.4, 71.5, 70.5, 38.1, 12.7

HRMS (ESI): calculated for C₂₀H₂₄N₂O₅Na [M+Na]⁺ 395.15774; found 395.1581. 2,6-Lutidine (2 eq., 713 μL, 5.62 mmol) is added to a solution of the compound 5 (1 eq., 1.046 g, 2.81 mmol) in a dioxane/H₂O mixture (3/1, 0.1 M, 28 mL). An osmium tetroxide solution (2.5 w % in tert-butanol, 0.03 eq., 851 μL, 0.084 mmol) and sodium periodate (3 eq., 2.14 g, 8.43 mmol) are added successively to the reaction medium. The white reaction medium is stirred at room temperature for 4 to 5 h. The reaction is stopped with water. The medium is extracted 3 times with dichloromethane. The organic phases are combined, washed with a saturated Na₂S₂O₅ solution, dried over Na₂SO₄, filtered and concentrated under reduced pressure. The reaction crude is purified by chromatography on silica gel (acetone/toluene 30/70 to 40/60 with a few drops of Et₃N) to obtain the compound 6 (747 mg, 71%, white foam).

Rf=0.28 (Toluene/Acetone 70/30)

IR (ATR) vₘₐₓ (cm⁻¹) 3,385, 3,202, 3,064, 3,038, 2,920, 2,872, 1,687, 1,468, 1,403, 1,357, 1,329, 1,276, 1,202, 1,134, 1,080, 962, 910, 773, 738, 700, 604, 560,492

NMR—¹H (300 MHZ, CDCl₃) δ (ppm) 9.67 (s, 1H), 9.26 (br. s, 1H), 7.57 (s, 1H), 7.41-7.25 (m, 5H), 6.37 (dd, J=6.0, 7.5 Hz, 1H), 4.55 (dd, J=11.7, 28.8 Hz, 2H), 4.33-4.27 (m, 1H), 4.26-4.16 (m, 3H), 3.81 (dd, J=2.7, 10.2 Hz, 1H), 3.67 (dd, J=3.0, 10.5 Hz, 1H), 2.46 (ddd, J=2.7, 6.0, 13.8 Hz, 1H), 2.22-2.09 (m, 1H), 1.88 (d, J=0.9 Hz, 3H)

NMR—¹³C (75.5 MHz, CDCl₃) δ (ppm) 198.0, 164.1, 150.6, 137.6, 136.0, 128.6, 128.6, 128.1, 127.8, 127.7, 111.2, 85.4, 83.6, 79.1, 77.4, 71.7, 71.6, 37.7, 12.6

HRMS (ESI): calculated for C₁₉H₂₂N₂O₆Na [M+Na]⁺ 397.1370; found 397.1368. The azolium salt of 3-ethyl-5-(2-hydroxyethyl)-4-methylthiazolium bromide (0.3 eq., 52 mg, 0.210 mmol), dried beforehand under vacuum for 1 day at 45° C., is loaded into a Schlenk tube and placed under argon. A solution of the compound 6 (1.5 eq., 384 mg, 1.02 mmol) in THF (0.5 M, 2.1 mL) is added to the Schlenk tube followed by distilled 1,8-diazabicyclo[5.4.0]undec-7-ene (DBU) (0.3 eq., 31 μL, 0.21 mmol). The medium becomes orange after the formation of the Breslow intermediate. The α-tocopherol derivative 9 (1.0 eq., 331 mg, 0.683 mmol) is added to the medium which is stirred at 75° C. for 18 h. After cooling to room temperature, the medium is concentrated under reduced pressure. The reaction crude is purified by silica gel chromatography (acetone/toluene 20/80) to obtain the compound 7 (126 mg, 29%, beige gum) in accordance with the invention.

Rf=0.49 (Acetone/Toluene 20/80)

IR (ATR) vₘₐₓ (cm⁻¹) 2,920, 2,852, 1,719, 1,690, 1,457, 1,423, 1,369, 1,330, 1,276, 1,252, 1,208, 1,157, 1,136, 1,082, 1,056, 1,008, 962, 936, 871, 780, 737, 699, 653, 611, 556, 493, 422

NMR—¹H (300 MHZ, CDCl₃) δ (ppm) 8.57 (brs, 1H), 7.66 (d, J=1.5 Hz, 1H), 7.40-7.27 (m, 5H), 6.38 (dd, J=5.7, 8.1 Hz, 1H), 6.55 (dd, J=13.5, 25.2 Hz, 2H), 4.35-4.18 (m, 4H), 3.78 (dd, J=2.7, 10.5 Hz, 1H), 3.63 (dd, J=3.0, 10.2 Hz, 1H), 3.02-2.93 (m, 2H), 2.79-2.69 (m, 2H), 2.62-2.52 (m, 2H), 2.46 (ddd, J=2.4, 5.7, 13.5 Hz, 1H), 2.19-2.10 (m, 1H), 2.07 (s, 3H), 2.00 (s, 3H), 1.96 (s, 3H), 1.89 (s, 3H), 1.85-1.66 (m, 3H), 1.61-0.98 (m, 26H), 0.92-0.79 (m, 12H)

NMR—¹³C (75.5 MHz, CDCl₃) δ (ppm) 205.3, 171.5, 164.0, 150.6, 149.6, 140.5, 137.6, 136.1, 128.6, 128.0, 127.8, 126.7, 124.9, 123.2, 117.5, 111.2, 85.3, 83.6, 79.3, 76.0, 75.2, 71.6, 71.6, 39.5, 37.7-37.4 (several aliphatic C: CH, CH₂), 33.2, 33.9, 32.8, 28.1, 27.4, 24.9, 24.5, 22.8, 22.7, 21.1, 20.7, 19.9, 19.8, 19.7, 13.1, 12.6, 12.2, 11.9

HRMS (ESI): calculated for C₅₁H₇₅O₉N₂ [M+H]⁺ 859.54671; found 859.54671.

### EXAMPLE 3

An active compound according to the invention (compound 14) is prepared according to the reaction scheme shown in FIG. 3, from the α-tocopherol derivative 9 prepared in Example 1, and the thymidine derivative compound 2 obtained as described in Example 2.

In a flask at 0° C., NaH (60% dispersion in an oil, 2.5 eq., 280 mg, 7.01 mmol) is added in portions in a solution of the compound 2 (1 eq., 1.00 g, 2.80 mmol) in THF (0.1 g/mL, 10 mL). The reaction medium is activated by microwaves (CEM machine, 200 W) at 40° C. for 4.5 min. The 2-methylnaphthalene bromide (2.5 eq., 1, 55 g, 7.01 mmol) is added to the reaction medium which is subjected afterwards to a second activation with microwaves (CEM machine, 200) at 40° C. for 2 h. After cooling to room temperature, the reaction is stopped with a saturated NH₄Cl solution. The medium is extracted 3 times with dichloromethane. The organic phases are combined, dried over Na₂SO₄, filtered and concentrated under reduced pressure. The reaction crude is purified by silica gel chromatography (EtOAc/pentane 30/70) to obtain the compound 10 (747.2 mg, 54%, colourless gum).

Rf=0.32 (EtOAc/pentane 30/70)

IR (ATR) vₘₐₓ (cm⁻¹) 2,926, 2,854, 1,735, 1,704, 1,644, 1,469, 1,377, 1,248, 1,146, 1,104, 998, 931, 781, 722, 619, 554, 496, 416.

NMR—¹H (300 MHZ, CDCl₃) δ (ppm) 9.56 (brs, 1H), 7.88-7.80 (m, 3H), 7.77 (s, 1H), 7.53-7.42 (m, 4H), 6.40 (dd, J=5.7, 8.7 Hz, 1H), 4.70 (dd, J=12.0, 30.9 Hz, 2H), 4.24-4.17 (m, 2H), 3.89 (dd, J=2.4, 11.4 Hz, 1H), 3.72 (dd, J=2.1, 11.4 Hz, 1H), 2.55 (dd, J=5.7, 13.2 Hz, 1H), 2.04-1.95 (m, 1H), 1.92 (s, 3H), 0.87 (s, 9H), 0.04 (d, J=8.4 Hz, 6H)

NMR—¹³C (75.5 MHz, CDCl₃) δ (ppm) 164.2, 150.6, 135.5, 135.0, 133.3, 133.1, 128.5, 127.9, 127.8, 126.7, 126.3, 126.1, 125.7, 111.0, 85.2, 85.1, 79.0, 71.5, 63.7, 38.1, 25.9, 18.4, 12.6, −5.3, −5.5.

At 0° C., a solution of tertbutylammonium fluoride in THF (1.0 M, 1 eq., 4.52 mL, 4.52 mmol) is added in drops to a solution of the compound 10 (1 eq., 2.25 g, 4.52 mmol) in THF (0.1 M, 45.2 mL). The medium is stirred at room temperature for 4.5 h. The reaction is stopped with water and then the medium is extracted 3 times with dichloromethane. The organic phases are combined, washed with brine, dried over Na₂SO₄, filtered and concentrated under reduced pressure. The reaction crude is purified by chromatography on silica gel ((CH₂Cl₂/MeOH 95/5) to obtain the compound 11 (1.43 g, 83%, white solid).

Rf=0.42 (EtOAc/Pentane 70/30)

IR (ATR) vₘₐₓ (cm⁻¹) 3,503.6, 3,166.8, 3,065.2, 3,023.7, 2,921.6, 1,709.0, 1,659.9, 1,508.0, 1,467.8, 1,398.2, 1,369.2, 1,346.4, 1,325.1, 1,281.6, 1,255.5, 1,225.5, 1,185.9, 1,058.8, 1,012.0, 974.5, 955.9, 906.5, 882.6, 859.2, 785.8, 768.2, 752.3, 653.2, 635.7, 608.7, 568.2, 549.5, 497.3, 441.8, 407.5

NMR—¹H (300 MHZ, dmso-d₆) δ (ppm) 11.35 (brs, 1H), 7.96-7.84 (m, 4H), 7.72 (d, J=1.5 Hz, 1H), 7.56-7.45 (m, 3H), 6.22 (dd, J=5.7, 8.4 Hz, 1H), 5.21-5.09 (m, 1H), 4.76-4.66 (m, 2H), 4.27-4.20 (m, 1H), 4.11-4.06 (m, 1H), 3.69-3.55 (m, 2H), 2.34 (ddd, J=2.4, 6.0, 13.8 Hz, 1H), 2.24-2.11 (m, 1H), 1.78 (d, J=1.2 Hz, 3H)

NMR—¹³C (75.5 MHz, dmso-d₆) δ (ppm) 163.8, 150.5, 136.0, 135.8, 132.8, 132.5, 127.9, 127.8, 127.6, 126.3, 126.0, 126.0, 125.9, 109.6, 84.8, 83.9, 79.1, 70.2, 61.6, 36.4, 12.3

HRMS (ESI): Calculated for C₂₁H₂₂O₅N₂Na [M+Na]⁺ 405.14209; found 405.1424. In a flask at 0° C., NaH (60% dispersion in an oil, 2.5 eq., 174 mg, 4.35 mmol) is added in portions in a solution of the compound 11 (1 eq., 669 mg, 1.74 mmol) in THF (0.1 g/mL, 6.7 mL). The reaction medium is activated by microwaves (CEM machine, 200 W) at 40° ° C. for 2 min. Allyl bromide (2.5 eq., 376 μL, 4.35 mmol) is added to the reaction medium which is subjected afterwards to a second activation with microwaves (CEM machine, 200) at 40° C. for 2 h. After cooling to room temperature, the reaction is stopped with a saturated NH₄Cl solution. The medium is extracted 3 times with dichloromethane. The organic phases are combined, dried over Na₂SO₄, filtered and concentrated under reduced pressure. The reaction crude is purified by silica gel chromatography (EtOAc/pentane 50/50) to obtain the compound 12 (607 mg, 83%, white solid).

Rf=0.40 (EtOAc/Pentane 50/50)

IR (ATR) vₘₐₓ (cm⁻¹) 2,932, 2,860, 1,731, 1,643, 1,462, 1,378, 1,249, 1,147, 1,106, 781, 622, 552, 419.

NMR—¹H (300 MHZ, Acetone-d₆) δ (ppm) 9.94 (brs, 1H), 7.94-7.86 (m, 4H), 7.69 (d, J=1.2 Hz, 1H), 7.57-7.46 (m, 3H), 6.38 (dd, J=6.0, 8.4 Hz, 1H), 6.02-5.87 (m, 1H), 5.30 (qd, J=1.8, 17.1 Hz, 1H), 5.17 (qd, J=1.5, 10.5 Hz, 1H), 4.84 (dd, J=12.0, 15.3 Hz, 2H), 4.42-4.37 (m, 1H), 4.33-4.27 (m, 1H), 4.10-4.05 (m, 2H), 3.72 (q, J=10.5 Hz, 1H), 3.71 (q, J=10.5 Hz, 1H), 2.48 (ddd, J=2.1, 5.7, 13.5 Hz, 1H), 2.31-2.20 (m, 1H), 1.80 (d, J=1.2 Hz, 3H)

NMR—¹³C 300 MHz, CDCl₃) δ (ppm) 164.0, 150.5, 136.0, 135.1, 134.0, 133.3, 133.2, 128.5, 128.0, 127.8, 126.6, 126.4, 126.2, 125.7, 117.6, 111.0, 85.3, 84.1, 79.3, 72.4, 72.4, 71.6, 70.5, 38.1, 12.7

HRMS (ESI): Calculated for C₂₄H₂₅O₅N₂ [M−H]⁻ 421.17635; found 421.17520. 2,6-lutidine (2 eq., 575 μL, 4.53 mmol) is added to a solution of the compound 12 (1 eq., 957 mg, 2.27 mmol) in a dioxane/H₂O mixture (3/1, 0.1 M, 22.7 mL). An osmium tetroxide solution (2.5 w % in tert-butanol, 0.03 eq., 694 μL, 0.068 mmol) and the sodium periodate (3 eq. 1, 45 g, 6.80 mmol) are added successively to the reaction medium. The white reaction medium is stirred at room temperature for 4 to 5 h. The reaction is stopped with water. The medium is extracted 3 times with dichloromethane. The organic phases are combined, washed with a saturated solution of Na₂S₂O₅, dried over Na₂SO₄, filtered and concentrated under reduced pressure. The reaction crude is purified by chromatography on silica gel (acetone/toluene 30/70 to 40/60 with a few drops of Et₃N) to obtain the compound 13 (634 mg, 66%, white foam).

Rf=0.24 (EtOAc/pentane 70/30)

IR (ATR) vₘₐₓ (cm⁻¹) 2,931, 2,858, 1,706, 1,647, 1,462, 1,249, 1,150, 1,109, 1,049, 1,000, 780, 624, 558, 417.

NMR—¹H (300 MHZ, CDCl₃) δ (ppm) 9.64 (s, 1H), 9.65 (brs, 1H), 7.88-7.75 (m, 4H), 7.57 (d, J=1.2 Hz, 1H), 7.53-7.42 (m, 3H), 6.40 (dd, J=5.7, 7.8 Hz, 1H), 4.73 (dd, J=12.0, 30.6 Hz, 2H), 4.38-4.31 (m, 1H), 4.28-4.23 (m, 1H), 4.20 (s, 2H), 3.81 (dd, J=2.7, 10.2 Hz, 1H), 3.66 (dd, J=2.7, 10.2 Hz, 1H), 2.50 (ddd, J=2.4, 6.0, 13.5 Hz, 1H), 2.23-2.11 (m, 1H), 1.89 (d, J=1.2 Hz, 3H)

NMR—¹³C (75.5 MHz, CDCl₃) δ (ppm) 197.9, 163.8, 150.5, 136.0, 135.0, 133.4, 133.2, 129.2, 128.5, 128.4, 128.0, 127.9, 126.7, 126.5, 126.3, 125.7, 125.4, 111.3, 85.4, 83.6, 79.1, 71.8, 37.7, 12.6

HRMS (ESI): Calculated for C₂₃H₂₃O₆N₂ [M−H]⁻ 423.15561; found 423.15449. Azolium salt of 3-ethyl-5-(2-hydroxyethyl)-4-methylthiazolium bromide (0.3 eq., 14 mg, 0.056 mmol), dried beforehand under vacuum for 1 day at 45° C., is loaded into a Schlenk tube and placed under argon. A solution of the compound 13 (1.5 eq., 118 mg, 0.279 mmol) in THF (0.5 M, 557 μL) is added to the Schlenk tube followed by distilled DBU (0.3 eq., 8.3 μL, 0.056 mmol). The medium becomes orange after the formation of the Breslow intermediate. The α-tocopherol derivative 9 (1.0 eq., 90 mg, 0.186 mmol) is added to the medium which is stirred at 75° C. for 48 h. After cooling to room temperature, the medium is concentrated under reduced pressure. The reaction crude is purified by silica gel chromatography (acetone/toluene 20/80) to obtain the compound 14 (9 mg, 5%, colourless gum) in accordance with the invention.

Rf=0.50 (Acetone/Toluene 20/80)

IR (ATR) vₘₐₓ (cm⁻¹) 2,926.0, 2,866.5, 1,687.1, 1,462.5, 1,410.9, 1,367.3, 1,274.2, 1,249.2, 1,196.2, 1,115.2, 1,079.9, 959.4, 913.4, 856.4, 817.4, 754.1, 618.0, 561.1, 476.5, 417.7

NMR—¹H (300 MHz, CDCl₃) δ (ppm) 8.19 (brs, 1H), 7.87-7.74 (m, 4H), 7.66 (s, 1H), 7.52-7.41 (m, 3H), 6.41 (dd, J=5.4, 7.8 Hz, 1H), 4.71 (dd, J=12.0, 27.6 Hz, 2H), 4.39-4.33 (m, 1H), 4.29-4.18 (m, 3H), 3.78 (dd, J=2.7, 10.2 Hz, 1H), 3.62 (dd, J=, 10.2 Hz, 1H), 2.94 (t, J=5.7 Hz, 2H), 2.70 (t, J=6.3 Hz, 2H), 2.60-2.44 (m, 3H), 2.20-2.11 (m, 1H), 2.07 (s, 3H), 1.99 (s, 3H), 1.95 (s, 3H), 1.89 (s, 3H), 1.84-1.67 (m, 4H), 1.59-0.97 (m, 26H), 0.89-0.78 (m, 12H)

NMR—¹³C (75.5 MHz, CDCl₃) δ (ppm) 205.2, 171.6, 163.6, 150.3, 149.7, 140.5, 136.2, 135.1, 133.4, 133.2, 128.5, 128.0, 127.9, 126.7, 126.4, 126.2, 125.7, 125.0, 123.3, 117.6, 111.2, 85.3, 83.8, 79.3, 76.0, 75.2, 71.7, 71.7, 39.5, 37.8-37.4 (several aliphatic C: CH, CH₂), 33.3, 32.9, 32.8, 28.1, 27.4, 24.9, 24.6, 22.9, 22.8, 21.2, 20.7, 19.9, 19.8, 13.1, 12.6, 12.2, 12.0.

### EXAMPLE 4

An active compound according to the invention (Compound 16) is prepared according to the reaction scheme shown in FIG. 4, starting from α-tocopherol 8 and from the compound 4 derived from thymidine obtained as described in Example 2.

4-dimethylaminopyridine (DMAP) (catalytic amount) and succinic anhydride (1 eq., 75 mg, 0.752 mmol) are added successively to a solution of the compound 4 (1 eq., 250 mg, 0.752 mmol) in toluene (0.044 M, 17.1 mL). The reaction medium is stirred under reflux for 5 h. After cooling to room temperature, the medium is concentrated under reduced pressure to obtain the compound 15. DMAP (0.6 eq., 17 mg, 0.138 mmol), the diacid compound 15 (1.5 eq., 149 mg, 0.345 mmol) and 1-ethyl-3-(3-dimethylaminopropyl) carbodiimide (EDC·HCl) (1.5 eq., 66 mg, 0.345 mmol) are added successively to a solution of α-tocopherol 8 (1 eq., 99 mg, 0.230 mmol) in dichloromethane (0.1 g/mL, 1 mL). The reaction medium is stirred at room temperature for 49 h The reaction is stopped with a saturated NH₄Cl solution. The medium is extracted 3 times with dichloromethane. The organic phases are combined, dried over Na₂SO₄, filtered and concentrated under reduced pressure. The reaction crude is purified by silica gel chromatography (EtOAc/pentane 40/60) to obtain the compound 16 (232 mg, 52% in 2 steps, white foam) in accordance with the invention.

Rf=0.10 (EtOAc/pentane 20/80)

IR (ATR) vₘₐₓ (cm⁻¹) 3181.2, 2926.5, 2867.1, 1744.0, 1688.5, 1457.0, 1412.0, 1365.5, 1273.9, 1249.8, 1203.7, 1145.0, 1105.5, 1060.7, 963.8, 911.5, 736.4, 698.7, 606.2, 558.2, 486.2, 419.2

NMR—¹H (300 MHz, CDCl₃) δ (ppm) 9.58 (brs, 1H), 7.40-7.27 (m, 6H), 6.32-6.24 (m, 1H), 4.57-4.39 (m, 3H), 4.31-4.22 (m, 2H), 4.15-4.08 (m, 1H), 3.03-2.92 (m, 2H), 2.81-2.72 (m, 2H), 2.63-2.55 (m, 2H), 2.49 (ddd, J=3.0, 6.0, 13.8 Hz, 1H), 2.12-2.00 (m, 7H), 1.98 (s, 3H), 1.92 (s, 3H), 1.85-1.67 (m, 2H), 1.63-1.00 (m, 26H), 0.93-0.78 (m, 12H)

NMR—¹³C (75.5 MHZ, CDCl₃) δ (ppm) 172.0, 171.0, 164.0, 150.4, 149.6, 140.5, 137.3, 135.2, 128.6, 128.1, 127.8, 126.6, 124.9, 123.1, 117.5, 111.3, 83.4, 82.2, 78.3, 75.1, 71.8, 64.2, 39.4, 37.7-37.4 (several C: CH, aliphatic CH₂), 32.8, 32.7, 31.0, 29.0, 28.7, 28.0, 24.9, 24.5, 22.8, 22.7, 21.1, 20.6, 19.8, 19.8, 19.7, 13.0, 12.7, 12.2, 11.9.

### EXAMPLE 5

A pharmaceutical composition C1 in accordance with the invention corresponds to the composition indicated in Table 1 hereinbelow.

TABLE 1

| Ingredient | Amount (mg) | % w/w |
|---|---|---|
| Compound 7 | 20 | 2 |
| α-tocopherol | 80 | 8 |
| Miglyol ® 812 | 100 | 10 |
| E80 Lecithins | 15 | 1.5 |
| Polysorbate 80 | 25 | 2.5 |
| Water | Q.S. 1,000 | 100 |

This composition is obtained as follows:
solubilising the compound 7 in accordance with the invention and the free α-tocopherol in the Miglyol® 812 oil, to form a lipophilic phase,
preparing an aqueous phase comprising at least the E80 lecithins (ionic surfactant) and polysorbate 80 (non-ionic surfactant),
separately heating the 2 phases at between 70 and 80° C.,
mixing by phase inversion by introducing the aqueous phase into the lipophilic phase at very high speed in an Ultra-Turrax® disperser so as to form a coarse emulsion, the lipid globules of which have an average diameter larger than 2 μm,
reducing the size of the lipid globules to obtain a nanoemulsion with a high-pressure homogeniser, a microfluidiser or an ultrasound probe in order to obtain that 100% of the globules have a size smaller than 1 μm with an average diameter comprised between 140 and 300 nm,
sterilising in an autoclave at 121° C. for 15 min or by sterilising filtration.

An oil-in-water nanoemulsion is obtained in which the prodrug according to the invention (compound 7) is in the lipophilic phase, in a perfectly solubilised form. The average diameter (average over 3 measurements) measured by dynamic light scattering (DLS) of the lipophilic phase droplets dispersed in the aqueous phase is 235 nm, for a polydispersity index PDI, measured by dynamic light scattering (DLS), of 0.23. The zeta potential, measured by electrophoretic light scattering (ELS), of this nanoemulsion in accordance with the invention is −38.3 mV, compatible with a satisfactory electrostatic repulsion for the stabilisation of the system.

This composition is particularly useful, inter alia, for the treatment of burns of chemical origin, in particular by topical application, and for the treatment of neurodegenerative diseases, in particular by oral or parenteral administration.

### EXAMPLE 6

A pharmaceutical composition C2 in accordance with the invention corresponds to the composition indicated in Table 2 hereinbelow.

TABLE 2

| Ingredient | Amount (mg) | % w/w |
|---|---|---|
| Compound 16 | 20 | 2 |
| Miglyol ® 812 | 200 | 20 |
| E80 Lecithins | 12 | 1.2 |
| Polysorbate 80 | 25 | 2.5 |
| Water | Q.S. 1,000 | 100 |

This composition is obtained by applying the protocol described in Example 5. An oil-in-water nanoemulsion is obtained in which the prodrug according to the invention (compound 16) is in the lipophilic phase, in a completely solubilised form.

For this nanoemulsion in accordance with the invention, are measured, on the same day (t=0 days), and then repeatedly over the next 28 days (t=1 day, 2 days, 3 days, 7 days, 14 days, 21 days, 28 days):
the average diameter of the lipophilic phase droplets dispersed in the aqueous phase, by dynamic light scattering (DLS);
the polydispersity index PDI, by dynamic light scattering (DLS);
and the zeta potential of the nanoemulsion, by electrophoretic light scattering (ELS).

The obtained results are indicated in Table 3 hereinbelow. In this table, each indicated value is the average of three measurements made.

TABLE 3

| | Diameter of | | |
|---|---|---|---|
| | lipophilic phase | | |
| Time | droplets dispersed in | | Zeta potential |
| (days) | the aqueous phase (nm) | PDI | (mV) |
| 0 | 157.9 +/− 0.6 | 0.101 +/− 0.012 | −48.9 +/− 1.4 |
| 1 | 159 +/− 2.1 | 0.113 +/− 0.013 | −40.9 +/− 0.4 |
| 2 | 159.3 +/− 1.6 | 0.087 +/− 0.014 | −40.9 +/− 0.3 |
| 3 | 157.8 +/− 2.3 | 0.11 +/− 0.013 | −26.0 +/− 1.6 |
| 7 | 159.1 +/− 1.7 | 0.11 +/− 0.021 | −19.4 +/− 1.1 |
| 14 | 159.8 +/− 3.4 | 0.105 +/− 0.024 | −37.5 +/− 1.3 |
| 21 | 160.6 +/− 1.7 | 0.1 +/− 0.001 | −38.9 +/− 1.1 |
| 28 | 161.5 +/− 1.7 | 0.098 +/− 0.012 | −33.7 +/− 1.2 |

It is observed that, for each of the 3 parameters, the obtained values are stable over time, which reflects a good stability of the nanoemulsion.

On average, the following results are obtained:
the average diameter of the lipophilic phase droplets dispersed in the aqueous phase is of about 160 nm;
with a polydispersity index always lower than 0.2;
and a zeta potential of about −40 mV.

This composition is particularly useful, inter alia, for the treatment of burns of chemical origin, in particular by topical application, and for the treatment of neurodegenerative diseases, in particular by oral or parenteral administration.

### EXAMPLE 7

Assessment of the Cytotoxicity of a Pharmaceutical Composition in the Form of a Nanoemulsion in Accordance with the Invention on Human Neuronal Cells

This experiment is carried out with the composition C2 of Example 6, containing the compound 16 as an active compound.

The following cell lines are used:
BE(2)-M17 (shScr-1, human dopaminergic neuroblastomas), obtained from ATCC® (reference CRL-2267™),
sh403-1 (human dopaminergic neuroblastoma) described in the publication by Dehay et al. PNAS, 2012, 109: 24, 9611-9616, featuring a stable inactivation (beta knockdown) of the ATPase ATP13A2 function.

These cell lines are cultured in an OPTI-MEM medium (Life Technologies, 31985-047) by adding 10% fetal bovine serum (Sigma-Aldrich) and 1% penicillin/streptomycin. For all experiments, freshly prepared nanoemulsions are used and the cells are cultured at 70-80% confluence. The cells are cultured for 24 or 48 h with the nanoemulsion diluted to 1,000th in the culture medium. An untreated control is also carried out. Each experiment is repeated at least three times. Cell viability is estimated by the MTS test (ATCC/LGC Promochem) according to the instructions of the manufacturer.

The obtained results are shown in FIG. 5. It is observed that the cell viability of the cells treated by the nanoemulsion in accordance with the invention (“NE”) is substantially equal to that of the untreated cells (“NT”), demonstrating the absence of toxicity of the active compound 16 according to the invention.

### EXAMPLE 8

An active compound according to the invention (Compound 18) is prepared according to the reaction scheme shown in FIG. 6, from fenchol 17 and the thymidine derivative 15 obtained as described in Example 4 from the compound 4.

The acid compound 15 (431 mg, 1.00 mmol, 1.5 eq.), DMAP (49 mg, 0.40 mmol, 0.6 eq.) and 1-ethyl-3-(3-dimethylaminopropyl)carbodiimide (EDC·HCl) (1.5 eq., 191 mg, 1.0 mmol are added successively to a solution of fenchol 17 (1 eq., 103 mg, 0.66 mmol) in dichloromethane (0.1 g/mL, 1 mL). The reaction medium is stirred at room temperature for 23.5 h. The reaction is stopped with a saturated NH₄Cl solution. The medium is extracted 3 times with dichloromethane. The organic phases are combined, dried over Na₂SO₄, filtered and concentrated under reduced pressure. The reaction crude is purified by chromatography on silica gel (EtOAc/pentane 50/50) to obtain the compound 18 (195 mg, 38% in 2 steps from 4, translucent gum).

Rf=0.36 (EtOAc/P 50/50);

IR (ATR) υₘₐₓ (cm⁻¹) 3,674, 2,961, 1,685, 1,456, 1,408, 1,379, 1,273, 1,201, 1,158, 1,046, 1,006, 909, 731, 697, 647, 604, 558, 488, 420;

NMR ¹H (300 MHz, CDCl₃) õ (ppm) 10.04 (s, 1H, NH), 7.40-7.16 (m, 6H), 6.26 (dd, J=6.9 Hz, 1H), 4.50 (AB system, J=11.7 Hz, 2H), 4.40-4.28 (m, 2H), 4.28-4.16 (m, 2H), 4.14-4.04 (m, 1H), 2.75-2.55 (m, 4H), 2.47 (ddd, J=3.0, 6.3, 13.8 Hz, 1H), 2.13-1.98 (m, 1H), 1.89 (s appearing, 3H), 1.75-1.56 (m, 3H), 1.56-1.47 (m, 1H), 1.46-1.19 (m, 2H), 1.18-1.10 (m, 1H), 1.04 (s, 3H), 0.99 (s, 3H), 0.72 (s, 3H);

NMR ¹³C (75.5 MHz, CDCl₃) 0 (ppm) 172.4, 171.9, 164.1, 150.5, 137.2, 135.2, 128.5, 127.9, 127.6, 111.1, 86.7, 85.2, 82.1, 78.2, 71.6, 64.1, 48.2, 48.1, 41.2, 39.3, 37.4, 29.6, 29.0, 28.9, 26.5, 25.7, 20.0, 19.3, 12.6.

### EXAMPLE 9

Comparative Example with No Lipidising Group

A compound which is not in accordance with the invention (compound 22), including no lipidising group in accordance with the invention, is prepared according to the reaction scheme shown in FIG. 7, starting from α-tocopherol 8 and from a thymidine derivative, 3′-O-tertbutyldiphenylsilylthymidine 19 obtained as described in the publication by Cunha et al., ACS Omega, 2020, 5, 11, 5815-5823.

### Synthesis of the Compound 19

The compound 19 has been obtained beforehand as follows:
1/ tert-butyldimethylsilyl chloride (TBDMSCl) (1,2 equiv, 4.48 g, 29, 72 mmol) and 4-dimethylaminopyridine (DMAP) (spatula tip) have been added sequentially to a solution of thymidine (1 equiv, 6 g, 24.77 mmol) in pyridine (118 mL) and under argon. The mixture has been stirred overnight at room temperature until completion of the reaction. Afterwards, the reaction has been stopped by the addition of NaHCO₃ (10 ml, aq.) and diluted with water (10 ml). The aqueous phase has been extracted three times with dichloromethane (DCM) (3×30 mL), and the combined organic phases have been dried over Na₂SO₄ before concentration to dryness under vacuum. The crude obtained has been purified by flash chromatography on silica gel (pentane/EtOAc, 70/30) to obtain a compound in the form of white powder (7.68 g, 21.55 mmol, 87%).
2/ imidazole (1.2 equiv, 3.37 g, 49.54 mmol) of tert-butylchlorodiphenylsilane (TBDPSCl) (1.2 equiv, 12.88 mL, 49.54 mmol) has been sequentially added to a solution of this compound (1 equiv, 14.7 g, 41.21 mmol) in dimethylformamide (DMF) (88 mL) and under argon. The mixture has been stirred overnight at room temperature until completion of the reaction. Afterwards, the mixture has been diluted with toluene (100 mL), and the DMF evaporated under vacuum to obtain a yellow oil. The oil has been diluted in diethyl ether (10 mL), and the formed white precipitate has been filtered and washed with an aqueous NaCl solution. The organic phase has been dried over Na₂SO₄ before concentration to dryness under vacuum. A compound has been obtained in the form of a yellow oil.
3/ para-toluenesulphonic acid monohydrate (0.471 g, 2.477 mmol) has been added to a solution of this compound in methanol (80 mL) and under argon. The mixture has been stirred overnight at room temperature until completion of the reaction. Afterwards, the methanol has been evaporated under vacuum to obtain a yellow oil. The crude product has been diluted with EtOAc (30 ml) and the organic phase washed three times with NaHCO₃ (10 ml, aq.) and brine (3×20 ml). The organic phase has been dried over Na₂SO₄ before concentration to dryness under vacuum. The resulting crude has been purified by flash chromatography on silica gel (petroleum ether/EtOAc, 50/50) to obtain the expected compound 19 in the form of a white powder (6.90 g, 14.36 mmol, 58% for steps 2/+3/).

Rf=0.29 (petroleum ether/EtOAc, 50/50);

NMR ¹H (300 MHZ, CDCl₃) δ ppm: 9.18 (bs, 1H, NH), 7.59-7.69 (m, 4H, H Ar), 7.34-7.50 (m, 6H, H Ar), 7.30 (d, J=1.2 Hz 1H, H6), 6.26 (dd, J=6.0 Hz, 7.8 Hz, 1H, H1′), 4.41-4.48 (m, 1H, H3′), 3.94-4.00 (m, 1H, H4′), 3.62 (dd, J=2.4 Hz, 12.0 Hz, 1H, H5b′), 3.24 (dd, J=3.0 Hz, 12.3 Hz, 1H, H5a′), 2.43 (bs, 1H, OH), 2.26 (ddd, J=3.0 Hz, 6.0 Hz, 13.2 Hz, 1H, H2b′), 2.04-2.22 (m, 1H, H2a′), 1.82 (d, J=1.2 Hz, 3H, H7), 1.08 (s, 9H, tBu);

NMR ¹³C (75.46 MHZ, CDCl₃) δ ppm: 164.0 (C4), 150.5 (C2), 136.9 (C6), 135.8 (CH Ar), 133.4 (Cq Ar), 133.3 (Cq Ar), 130.2 (CH Ar), 130.2 (CH Ar), 128.0 (CH Ar), 111.0 (C5), 87.8 (C4′), 86.6 (C1′), 73.1 (C3′), 62.1 (C5′), 40.4 (C2′), 27.0

(CH3tBu), 19.1 (Cq tBu), 12.5 (C7);

IR (ATR) v (cm⁻¹): 2,932, 1,682, 1,471, 1,274, 1,104, 1,031, 740, 702.

### Synthesis of the Compound 22

DMAP (catalytic amount) and succinic anhydride (1 eq., 42.4 mg, 0.424 mmol) are added successively to a solution of the compound 19 (1 eq., 204 mg, 0.424 mmol) in toluene (0.1 M, 4.24 mL). The reaction medium is stirred under reflux for 6 h. After cooling to room temperature, the medium is concentrated under reduced pressure to obtain the compound 20 which is engaged in the following reaction with no subsequent purification.

DMAP (0.6 eq., 20.7 mg, 0.169 mmol), the compound 20 (1.5 eq., 0.424 mmol) and the EDC·HCl (1.5 eq., 81 mg, 0.424 mmol) are added successively to a solution of α-tocopherol 8 (1 eq., 121.7 mg, 0.282 mmol) in dichloromethane (0.1 g/mL, 1 mL). The reaction medium is stirred at room temperature for 48 h. The reaction is stopped with a saturated NH₄Cl solution. The medium is extracted 3 times with dichloromethane. The organic phases are combined, dried over Na₂SO₄, filtered and concentrated under reduced pressure. The reaction crude containing the compound 21 is used with no subsequent purification in the next reaction.

Tetrabutylammonium fluoride TBAF (c=1M in THF, 1 eq. 282 μl, 0.282 mmol) is added to a solution of the compound 21 (0.282 mmol) at 0° C. Once the temperature has reached room temperature, the reaction is stirred for 2 h. The reaction is stopped with H₂O. The medium is extracted 3 times with dichloromethane. The organic phases are combined, dried over Na₂SO₄, filtered and concentrated under reduced pressure. The reaction crude is purified by chromatography on silica gel (toluene/acetone 70/30) to obtain the compound 22 (177 mg, 45% over 3 white solid steps).

Rf=0.25 (toluene/acetone 70/30);

IR (ATR) vₘₐₓ (cm⁻¹) 3,456, 3,178, 2,926, 2,867, 1,742, 1,686, 1,461, 1,412, 1,373, 1,242, 1,145, 1,100, 914, 736, 695, 606, 489, 417;

NMR ¹H (300 MHZ, CDCl₃) δ (ppm) 9.76 (brs, 1H), 7.30 (s, 1H), 6.28 (t, 1H), 4.43 (dd, J=3.0, 12 Hz, 1H), 4.34 (brs, 1H), 4.27 (dd, J=3.0, 12 Hz, 1H), 4.17-4.07 (m, 1H), 3.63 (brs, 1H), 3.03-2.91 (m, 2H), 2.84-2.71 (m, 2H), 2.63-2.51 (m, 2H), 2.47-2.34 (m, 1H), 2.19-2.04 (m, 1H), 2.07 (s, 3H), 2.0 (s, 3H), 1.96 (s, 3H), 1.89 (s, 3H), 1.85-1.65 (m, 2H), 1.63-0.97 (m, 26H), 0.93-0.76 (m, 12H);

NMR ¹³C (75.5 MHz, CDCl₃) δ (ppm) 172.2, 171.2, 164.2, 150.8, 149.6, 140.5, 137.5, 126.7, 124.9, 123.1, 117.5, 111.4, 85.2, 84.3, 75.2, 71.4, 64.3, 40.2, 39.4, 37.6, 37.5, 37.5, 37.3, 32.8, 32.8, 29.1, 28.7, 28.0, 24.9, 24.5, 22.8, 22.7, 21.1, 20.6, 19.8-19.7 (several aliphatic C: CH, CH₂), 13.0, 12.6, 12.2, 11.9.

### EXAMPLE 10

### Solubility in Oil

The compound 16 in accordance with the invention and the comparative compound 22, devoid of a lipidising group, are subjected to a solubility test in Miglyol® 812, at different concentrations between 1 and 5% w/w

The results are shown in FIG. 8. As one can observe, in a/ in this figure, at the concentration of 5% w/w, the compound 16 in accordance with the invention is totally soluble in the oily vehicle. The comparative compound 22 proves to be insoluble: at the concentration of 1% w/w, in b/ in the figure, one can observe the presence of an interface in the medium, indicated by an arrow, indicating a non-miscibility of the phases in presence. This phenomenon is even more clear at concentrations of 2% w/w and of 5% w/w shown in c/ and in d/ respectively in the figure. This result demonstrates the impossibility of formulating the comparative compound 22 in a composition in the particularly advantageous form of a nanoemulsion, unlike the compound 16 in accordance with the invention.

### EXAMPLE 11

### UV Irradiation

This test has been carried out on the compound 16 in accordance with the invention, by means of a light bulb simulating the sunlight Osram Ultra Vitalux 300 W 230 V, with an UVA radiation in the range from 315 to 400 nm (13.6 W) and UVB in the range from 280 to 315 nm (3.0 W).

The compound has been placed in the deuterated solvent CDCl₃ (30 mg in 750 μl) to follow the reaction by NMR ¹H. Before irradiation, the signals of the methyl groups (CH₃) of the tocopherol skeleton and of the nucleobase and the anomeric signal H₁′ of the nucleoside have a normal multiplicity, as shown in FIG. 9: 4 singlets for the methyl groups and a triplet for the anomer. After 8 h of irradiation, as shown in FIG. 10, a doubling of the signals of these methyl groups and of the anomeric signal of the nucleoside reveals superimposed signals reflecting the presence of two other compounds, derived from the cleavage, induced by the ultraviolet radiation, of the ester functions, as indicated by zigzag lines in the figure.

These results presage an even more rapid cleavage of the compound, to release α-tocopherol, within a nanoemulsion, i.e. in the presence of water, a good nucleophile promoting the breakage of the ester bond.

### EXAMPLE 12

### Study of the Antioxidant Effect on Keratinocytes

This study has been carried out on the above-described compound 16 in accordance with the invention.

Two pharmaceutical compositions C3 and C4 in accordance with the invention meeting the compositions indicated in Table 4 hereinbelow have been prepared.

TABLE 4

| Ingredient | C3 (% w/w) | C4 (% w/w) |
|---|---|---|
| Compound 16 | 3.0 | 5.0 |
| E80 Lecithins | 1.2 | 1.2 |
| Miglyol ® 812 | 15.8 | 13.8 |
| Tween ® 80 | 2.5 | 2.5 |
| Glycerol | 2.3 | 2.3 |
| Water Milli-Q ® or p.p.i. | Q.S. 100 | Q.S. 100 |

To this end, the following protocol has been implemented.

An oily phase has been prepared by dispersing the E80 lecithins and the compound 16 in the Miglyol® 812 under ultrasounds at 70° C., until complete dispersion. An aqueous phase has been prepared by dispersing Tween® 80 and glycerol in Milli-Q® water or for injectable preparation (p.p.i.). A phase inversion has been carried out with 800 mg of the aqueous phase and 200 mg of the oily phase. The mixture has been vortexed and then sonicated with a probe for 8 min.

The assessment of the antioxidant effect of the compositions C3 and C4 according to the invention has been based on the dosage of the amount of reactive oxygen species (ROS) produced in the medium by flow cytometry, after setting the cells treated with these compositions in presence with the oxidative stress indicator CM-H₂DCFDA (5-(and-6)-chlorométhyl-2′,7′-dichlorodihydrofluorescein diacetate, acetyl ester) (Invitrogen®). By cleavage of its acetate groups with esterases and its intracellular oxidation by the ROS, this non-fluorescent indicator is converted into highly-fluorescent dichlorofluorescein 2′,7′ (DCF). Thus, the measurement of the fluorescence intensity allows deducing the amount of DCF, and consequently of ROS, in the cell medium. Primary keratinocytes isolated from mammoplasty and immortalised by introduction and overexpression of the gene of the reverse transcriptase of the telomerase hTERT and introduction of the antigen T of the SV40 virus, have been used for these experiments. The culture medium is the medium KGM2 (Keratinocyte Growth Medium 2) (Promocell®), and the culture temperature 37° C.

In a 1^{st} experiment, cells have been incubated with the composition C4 diluted to 1/1,000^{th} in the culture medium for 30 min. Afterwards, they have been rinsed with phosphate-buffered saline (PBS), then irradiated with a solar simulator (Suntest CPS+, Atlas) (300-800 nm), in order to induce ROS production, for 2 min at 765 W/m² (90 K) in PBS added with the composition C4 diluted to 1/1,000^{th}. Finally, they have been incubated again with the composition C4 diluted to 1/1,000^{th} in the culture medium for 30 min. A negative control has been carried out in the same manner, without the composition C4.

Afterwards, the cells have been detached by tryptinisation by means of trypsin at 0.0025% and EDTA at 0.01% for 10 min at 37° C., neutralised by fetal calf serum at 10% and separated from the medium by centrifugation. They have been incubated for 20 min at 37° C. with the CM-H₂DCFDA probe at a final concentration of 1 M (incubation volume 100 μl). Afterwards, the fluorescence has been read by a flow cytometer Accuri® 6.

The obtained results, expressed in % of mean fluorescence intensity (MFI) with respect to the non-irradiated (and without C4) control condition, are shown in FIG. 11. It is observed that the composition C4 according to the invention inhibits the production of ROS (decrease in the intensity of the fluorescence, reflecting a decrease in the DCF production from CM-H₂DCFDA), while without this composition the irradiation induces a production of ROS in the range of 20%. In a 2^{nd} experiment, cells have been incubated with the composition C4 diluted to 1/1,000^{th} in the culture medium, the composition C3 diluted to 1/1,000^{th} in the culture medium, or α-tocopherol at 0.5% w/v in the culture medium, for 48 h. Afterwards, the cells have been treated as described hereinabove, with the composition C4 diluted to 1/1,000^{th} in the culture medium, the composition C3 diluted to 1/1,000^{th} in the culture medium, or α-tocopherol at 0.5% w/v in the culture medium, for irradiation and post-irradiation incubation.

The obtained results, expressed in % of mean fluorescence intensity (MFI) with respect to the control condition without additive and not irradiated, are shown in FIG. 12. α-tocopherol at 0.5% w/v, an antioxidant positive control at this concentration, strongly inhibits the production of ROS induced by the UV radiation as well as intracellular ROS. The composition C3 according to the invention partially inhibits the production of ROS induced by the ultraviolet radiation. The composition C4 according to the invention, more concentrated in active compound, strongly inhibits the production of UV-induced ROS, as well as cytoplasmic ROS.

All of these results demonstrate a good antioxidant efficiency of the composition C4 in accordance with the invention, as well as, to a lesser extent, of the composition C3 in accordance with the invention.

What is claimed is:

1. A prodrug, consisting of:
an active compound comprising:
a pharmaceutically-active antioxidant molecule covalently coupled, by a bond comprising at least one carboxylic ester group, to a nucleoside comprising a ribose or deoxyribose unit covalently bonded to a nucleic base,
said nucleoside bearing at least one group, referred to as a lipidising group, selected in the group consisting of the groups —R and —CO—R, wherein R represents a linear, branched and/or cyclic, saturated and/or unsaturated, aromatic or non-aromatic, hydrocarbon radical, optionally substituted, optionally interrupted by one or more heteroatom(s) and/or by one or more group(s) comprising at least one heteroatom or at least one group comprising at least one heteroatom, R comprising no hydroxyl, primary amine, secondary amine, phosphate or thiol groups, and possibly including one cycle or several cycles, optionally condensed,
each of the hydroxyl groups of said ribose or deoxyribose unit not involved in a bond to said antioxidant molecule or to a lipidising group being substituted by a protective group of a hydroxyl function attached to the oxygen atom of said hydroxyl group and selected in the group consisting of linear, branched and/or cyclic, saturated and/or unsaturated, aromatic or non-aromatic, hydrocarbon radicals, optionally substituted, optionally interrupted by one or more heteroatom(s) and/or by one or more group(s) comprising at least one heteroatom or at least one group comprising at least one heteroatom, excluding hydroxyl, primary amine, secondary amine, phosphate and thiol groups, and possibly including one cycle or several cycles, where appropriate one or more heterocycle(s) comprising one or more heteroatom(s), said cycles being optionally condensed,
and each of the primary amine groups not involved in a bond to said antioxidant molecule or to a lipidising group being substituted by a protective group of a primary amine function attached to the nitrogen atom of said primary amine group and selected from the group consisting of linear, branched and/or cyclic, saturated and/or unsaturated, aromatic or non-aromatic, hydrocarbon radicals, optionally substituted, optionally interrupted by one or more heteroatom(s) and/or by one or more group(s) comprising at least one heteroatom or at least one group comprising at least one heteroatom, excluding hydroxyl, primary amine, secondary amine, phosphate and thiol groups, and which may include one cycle or several cycles, optionally one or more heterocycle(s) comprising one or more heteroatom(s), said cycles being optionally condensed,
or one of the pharmaceutically-acceptable salts of said active compound.

2. The prodrug according to claim 1, wherein said lipidising group is selected from the group consisting of:
the groups —CH₂—R′, —CO—CH₂—R′ and —CO—(CH₂)₂—CO—O—R′, where R′ represents cholesterol, tocopherol, tocotrienol, a phenyl ring possibly substituted by one or more group(s) R₅, a naphthyl ring possibly substituted by one or more group(s) R₅, or a pyridine ring possibly substituted by one or more group(s) R₅, wherein R₅ represents a linear or branched, saturated or unsaturated, C1-C25 hydrocarbon chain,
an allyl radical or a propargyl radical,
the groups R and —CO—R, where R represents: a linear or branched, saturated or unsaturated, C1-C25 hydrocarbon chain; a group —(CH₂)₂—O—R₅—O—CH₃ where R₅ represents a linear or branched, saturated or unsaturated, C1-C25 hydrocarbon chain; a group —CH₂—CH(O—R₆)—O—R₇ where Re and R₇, identical or different, each represent a linear or branched, saturated or unsaturated, C1-C25 hydrocarbon chain; a group —CH(O—R₆)—O—R₇ where R₆ and R₇, identical or different, each represent a linear or branched, saturated or unsaturated, C1-C25 hydrocarbon chain; or a group —CH₂—CH(O—CO—R₆)—O—CO—R₇ where R₆ and R₇, identical or different, each represent a linear or branched, saturated or unsaturated, C1-C25 hydrocarbon chain,
the groups —CH₂—R₈ where R₈ represents a triazole group, a benzothiophene group, a benzofuran group or an indole group, R₈ being optionally substituted by one or more group(s) R₅ where R₅ represents a linear or branched, saturated or unsaturated, C1-C25 hydrocarbon chain,
the groups —(CH₂)₂—O—(CH₂)₂—O—(CH₂)ₚ—O—CH₂—R₁₅, where p is an integer comprised between 2 and 25 and R₁₅ represents a hydrogen atom or a phenyl group;
the groups —CO—CH₂—O—(CH₂)_{q}—O—CH₂—R₁₆, where q is an integer comprised between 2 and 25 and R₁₆ represents a hydrogen atom or a phenyl group; and
a triazole group, optionally substituted by a linear or branched, saturated or unsaturated, C1-C25 hydrocarbon chain.

3. The prodrug according to claim 1, wherein the coupling of said antioxidant molecule to said nucleoside is carried out on a first oxygen atom of said nucleoside selected from the group consisting of the oxygen atom located in the position 5′ of said ribose or deoxyribose unit and the oxygen atom located in the position 3′ of said ribose or deoxyribose unit.

4. The prodrug according to claim 3, wherein said lipidising group is carried by a second oxygen atom of said nucleoside, different from said first oxygen atom, selected from the group consisting of the oxygen atom located in the position 5′ of said ribose or deoxyribose unit and the oxygen atom located in the position 3′ of said ribose or deoxyribose unit, and/or by a nitrogen atom of an amine group of said nucleic base.

5. The prodrug according to claim 1, wherein said nucleoside has the general formula (I): 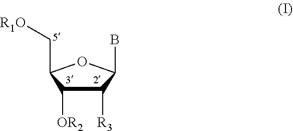
wherein
B represents said nucleic base, possibly substituted at an amine group by a lipidising group, each of the possible primary amine groups of said nucleic base not involved in a bond to said antioxidant molecule or to a lipidising group being substituted by a protective group of a primary amine function as defined in claim 1,
and
either R₁ represents the bond to said antioxidant molecule, and
R₂ represents a lipidising group,
R₃ represents H or OR₄ where R₄ represents a lipidising group or a protective group of a hydroxyl function as defined in claim 1,
or R₂ and R₄ form together, with the oxygen atoms to which they are attached, an acetal group comprising one or more linear, branched and/or cyclic, saturated or unsaturated, aromatic or non-aromatic, hydrocarbon chain(s), optionally substituted, optionally interrupted by one or more heteroatom(s) and/or by one or more group(s) comprising at least one heteroatom or at least one group comprising at least one heteroatom, excluding hydroxyl, primary amine, secondary amine, phosphate and thiol groups, and possibly including one or more cycle(s), optionally one or more heterocycle(s) comprising one or more heteroatom(s), the cycles being optionally condensed,
or R₁ represents the bond to said antioxidant molecule, and
R₂ represents a lipidising group or a protective group of a hydroxyl function as defined in claim 1,
R₃ represents OR₄ where R₄ represents a lipidising group,
or R₂ represents the bond to said antioxidant molecule, and
R₁ represents a lipidising group,
R₃ represents H or OR₄ where R₄ represents a lipidising group or a protective group of a hydroxyl function as defined in claim 1,
or R₂ represents the bond to said antioxidant molecule, and
R₁ represents a lipidising group or a protective group of a hydroxyl function as defined in claim 1,
R₃ represents OR₄ where R₄ represents a lipidising group.

6. The prodrug according to claim 1, wherein said nucleic base of said nucleoside is a natural nitrogenous base selected from the group consisting of adenine, guanine, uracil, thymine and cytosine, optionally substituted, or a non-natural monocyclic or bicyclic heterocyclic base, each cycle of which includes from 4 to 7 members.

7. The prodrug according to claim 1, wherein said antioxidant molecule is covalently coupled to said nucleoside via a spacer arm containing at least one carboxylic ester group and/or at least one carbonyl group bonded to the oxygen atom at the position 5′ of said ribose or deoxyribose unit or to the oxygen atom at the position 3′ of said ribose or deoxyribose unit or to an oxygen atom of said antioxidant molecule.

8. The prodrug according to claim 1, wherein said antioxidant molecule is covalently coupled to said nucleoside via a spacer arm of general formula (II): 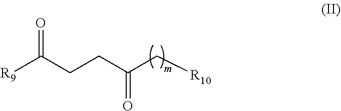
wherein R₉ represents the antioxidant molecule and R₁₀ represents the nucleoside, and
m is equal to 0 and bonding of the antioxidant molecule to the spacer arm is carried by an oxygen atom of the antioxidant molecule and/or bonding of the nucleoside to the spacer arm is carried by an oxygen atom of the nucleoside,
or m is equal to 1 and bonding of the antioxidant molecule to the spacer arm is carried by an oxygen atom of the antioxidant molecule.

9. The prodrug according to claim 1, wherein said antioxidant molecule is selected from the group consisting of α-tocopherol, β-tocopherol, γ-tocopherol, α-tocotrienol, β-tocotrienol, γ-tocotrienol, δ-tocotrienol, resveratrol, lipoic acid, retinol, retinal, retinoic acid and ascorbic acid.

10. The prodrug according to claim 1, of general formula (IIIa): 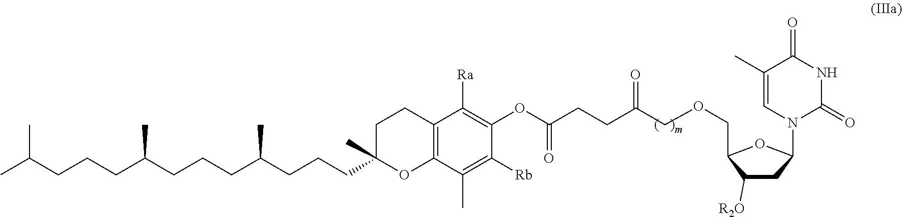
wherein
m is equal to 0 or 1,
Ra and Rb, identical or different, each represent a hydrogen atom or a methyl group,
and R₂ represents said lipidising group.

11. A pharmaceutical composition containing a prodrug according to claim 1 in a pharmaceutically-acceptable carrier.

12. The pharmaceutical composition according to claim 11, in the form of an emulsion comprising a lipophilic phase and an aqueous phase, said prodrug being contained in said lipophilic phase.

13. The pharmaceutical composition according to claim 12, comprising from 1 to 35% by weight of said lipophilic phase, with respect to the total weight of said composition.

14. The pharmaceutical composition according to claim 11, comprising between 0.1 and 10% by weight of said prodrug, with respect to the total weight of the composition.

15. The pharmaceutical composition according to claim 11, further comprising a pharmaceutically-active antioxidant molecule in free form.

16. A method of prophylactically or curatively treating a disease in a subject in need thereof, comprising administering to said subject a therapeutically-effective amount of a prodrug according to claim 1.

17. The method according to claim 16, wherein said prodrug is administered to treat chemical or radiological burns.

18. The method according to claim 17, wherein said prodrug is administered topically to said subject.

19. The method according to claim 16, wherein said prodrug is administered to treat a neurodegenerative disease.

20. The method according to claim 19, wherein said prodrug is administered orally or parenterally to said subject.
